(19) United States
(12) Patent Application Publication
(10) Pub. No.: US 2025/0356944 A1
(43) Pub. Date: Nov. 20, 2025

(54) GENERATING A MECHANISM OF ACTION REPRESENTATION FROM CELL REPRESENTATION EMBEDDINGS TO PREDICT A MECHANISM OF ACTION FOR A PERTURBATION

(71) Applicant: Recursion Pharmaceuticals, Inc., Salt Lake City, UT (US)

(72) Inventors: Alex Fogli Iseppe, Davis, CA (US); Aurora Skye Blucher, Salt Lake City, UT (US); Benjamin Marc Feder Fogelson, Salt Lake City, UT (US); Jacob Carter Cooper, Sandy, UT (US); Kyle Rollins Hansen, Kaysville, UT (US); Marissa Gerda Saunders, Salt Lake City, UT (US); Marta Marie Fay, Salt Lake City, UT (US); Nathan Henry Lazar, Salt Lake City, UT (US); Rachel Jie Min Ng, San Francisco, CA (US); Safiye Celik, Sudbury, MA (US); Thomas Arian Sasani, Atlanta, GA (US)

(21) Appl. No.: 18/663,819

(22) Filed: May 14, 2024

(51) Int. Cl. G16B 5/00; G16B 40/20

(52) U.S. Cl. CPC G16B 5/00; G16B 40/20

(57) ABSTRACT

The present disclosure relates to systems, non-transitory computer-readable media, and methods for deducing information for mechanism of actions (MOAs) utilizing digital signals from cell representations within a shared feature space. In particular, the disclosed systems can deduce (or predict) MOAs by generating MOA representations with corresponding detection confidence scores that indicate whether cell representations in a MOA representation provide a meaningful signal to predict the MOA. Indeed, the disclosed systems can determine a cluster of cell representation embeddings (in the shared feature space) based on annotated cell representation embeddings corresponding to a known MOA to generate an MOA representation. Furthermore, the disclosed systems can utilize MOA representations, within the shared feature space, to predict MOAs for a query cell representation (of a perturbation). Moreover, the disclosed systems can also generate a measure of confidence (that the query perturbation exhibits the predicted MOA (from the MOA representation).

### BACKGROUND

In recent years, there have been significant improvements in hardware and software platforms for utilizing computing devices to extract and analyze digital signals corresponding to biological relationships. For instance, existing systems often utilize computer-based models to extract latent features from images portraying cells. In addition, such existing systems often conduct analyses of the features extracted from cell images to determine biological (or chemical) relationships from the images. Indeed, existing systems often infer biological relationships from cellular phenotypes in high-content microscopy screens by using deep vision models to capture biological signals. Although conventional systems can utilize computer-based models to extract and analyze digital signals for images portraying cells, these conventional systems often have a number of technical shortcomings with regard to inflexible and inefficient utilization of the extracted microscopy features (or digital signals) and inaccurate predictions of certain biological relationships from the extracted microscopy features (or digital signals).

### SUMMARY

Embodiments of the present disclosure provide benefits and/or solve one or more of the foregoing or other problems in the art with systems, non-transitory computer-readable media, and computer-implemented methods for deducing information for mechanism of actions (MOAs) utilizing digital signals from cell representations within a shared feature space. In particular, the disclosed systems can deduce (or predict) MOAs by generating MOA representations with corresponding detection confidence scores that indicate whether cell representations in a MOA representation provide a meaningful signal to predict the MOA. For example, the disclosed systems can access MOA annotation data (e.g., known MOAs for particular genes or compounds) and annotate cell representation embeddings (e.g., phenomic image representation embeddings) that correspond to the known MOAs in a shared feature space. In addition, the disclosed systems can determine a cluster of cell representation embeddings (in the shared feature space) based on the annotated cell representation embeddings to generate an MOA representation. Moreover, the system can determine a mechanism of action detection confidence score by comparing whether the cell representation signals in the MOA representation provide a more accurate signal relative to a plurality of sampled cell representations outside of the embedding cluster of the MOA representation.

In addition, the disclosed systems can utilize MOA representations, within the shared feature space, to predict MOAs for a query cell representation (of a perturbation). For instance, the disclosed systems can determine similarity measures between one or more MOA representations and an embedding of the query cell representations to generate a predicted MOA for the query perturbation. Moreover, in one or more instances, the disclosed system also generates a measure of confidence (i.e., a confidence score) that the query perturbation exhibits the predicted MOA. Indeed, the disclosed systems can generate a confidence score by comparing the similarity measure between the MOA representation and the query perturbation against similarity measures between the MOA representation and other sampled query cell representations. Furthermore, the disclosed systems can display user interfaces to display visualizations of MOA representations and MOA detection confidence scores and/or predicted MOAs for queries and corresponding confidence scores.

Additional features and advantages of one or more embodiments of the present disclosure are outlined in the description which follows, and in part can be determined from the description, or may be learned by the practice of such example embodiments.

### BRIEF DESCRIPTION OF THE DRAWINGS

The detailed description is described with reference to the accompanying drawings in which:

FIG. 1 illustrates an overview of a mechanism-of-action detection system generating MOA representations in accordance with one or more embodiments.

FIG. 2 illustrates an overview of a mechanism-of-action detection system predicting a mechanism of action for a mechanism of action query in accordance with one or more embodiments.

FIG. 3 illustrates a mechanism-of-action detection system identifying and annotating cell representation embeddings with mechanism of actions in accordance with one or more embodiments.

FIG. 4 illustrates a mechanism-of-action detection system generating mechanism of action representations from annotated cell representation embeddings in accordance with one or more embodiments.

FIG. 5 illustrates a mechanism-of-action detection system generating an MOA detection confidence score for an MOA representation in accordance with one or more embodiments.

FIG. 6 illustrates a mechanism-of-action detection system predicting a mechanism of action for a query in accordance with one or more embodiments.

FIG. 7 illustrates a mechanism-of-action detection system generating a prediction confidence score for a predicted mechanism of action in accordance with one or more embodiments.

FIG. 8 illustrates a mechanism-of-action detection system generating predicted MOAs for compound clusters determined from a query list of compounds in accordance with one or more embodiments.

FIG. 9 illustrates a mechanism-of-action detection system displaying an exemplary graphical user interface for an MOA representation in accordance with one or more embodiments.

FIG. 10 illustrates a mechanism-of-action detection system displaying an exemplary graphical user interface for an MOA query and MOA predictions for the MOA query in accordance with one or more embodiments.

FIG. 11 illustrates a mechanism-of-action detection system displaying an exemplary graphical user interface for predicted MOAs of compound clusters in accordance with one or more embodiments.

FIG. 12 illustrates a schematic diagram of an exemplary environment of a tech-bio exploration system and a mechanism-of-action detection system in accordance with one or more embodiments.

FIG. 13 illustrates a schematic diagram of a system environment in which a mechanism-of-action detection system can operate in accordance with one or more embodiments.

FIG. 14 illustrates an example series of acts for generating cell representations within a shared feature space in accordance with one or more implementations herein.

FIG. 15 illustrates a block diagram of an example computing device for implementing one or more embodiments of the present disclosure.

### DETAILED DESCRIPTION

This disclosure describes one or more embodiments of a mechanism-of-action detection system that generates mechanism of action (MOA) representations utilizing digital signals from cell representations within a shared feature space that enable MOA predictions for query perturbations. For instance, the mechanism-of-action detection system can identify cell representation embeddings generated utilizing a machine learning model (e.g., a machine learning model that is trained to predict perturbations from cell representations or generate predicted cell representations from masked cell representations). In addition, the mechanism-of-action detection system can annotate the cell representation embeddings with MOAs that correspond with the cell representation embeddings. Moreover, the mechanism-of-action detection system can generate an MOA representation from an embedding cluster that represents the annotated cell representation embeddings within a shared feature space. In addition, the mechanism-of-action detection system can also determine an MOA detection confidence score for the MOA representation that indicates whether the annotated cell representation embeddings provide a meaningful signal for deducing a particular MOA in comparison to embeddings outside of the MOA representation.

Additionally, the mechanism-of-action detection system can also utilize MOA representations, within the shared feature space, to predict MOAs for a query cell representation (of a perturbation). In particular, the mechanism-of-action detection system can receive (or identify) an MOA query for a particular perturbation. In response, the mechanism-of-action detection system can identify a query cell representation embedding (for the particular perturbation) for the shared feature space (e.g., an embedding generated by the above-mentioned machine learning model). Moreover, the mechanism-of-action detection system can generate a predicted MOA for the perturbation related to the MOA query based on a comparison of the query cell representation embedding with the MOA representation (e.g., via similarity measures in the shared feature space). In addition, the mechanism-of-action detection system can also determine a confidence score for the predicted MOA that indicates a measure of confidence that the query cell representation exhibits the predicted MOA.

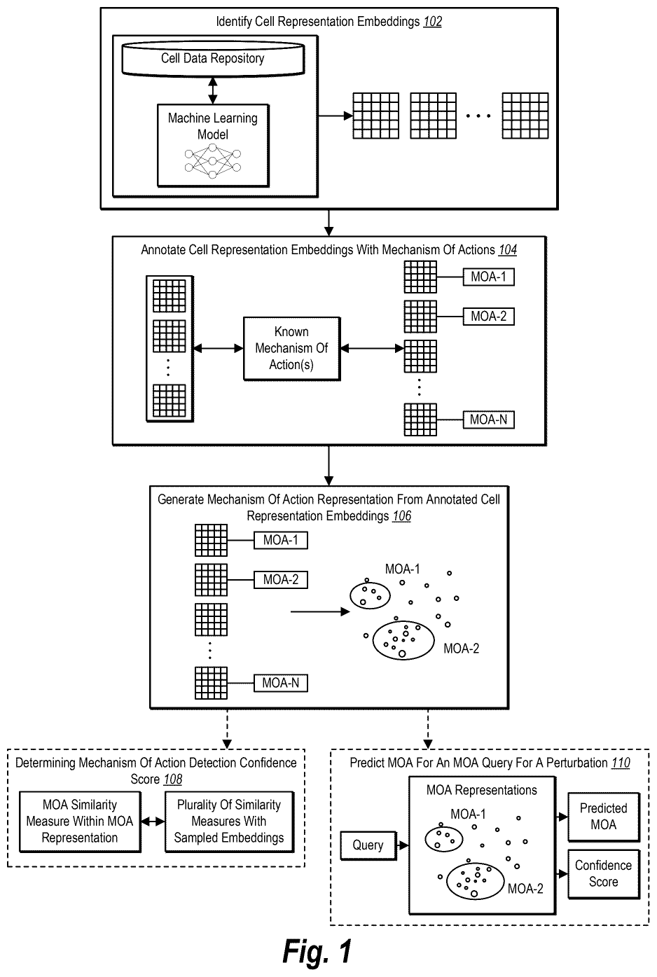

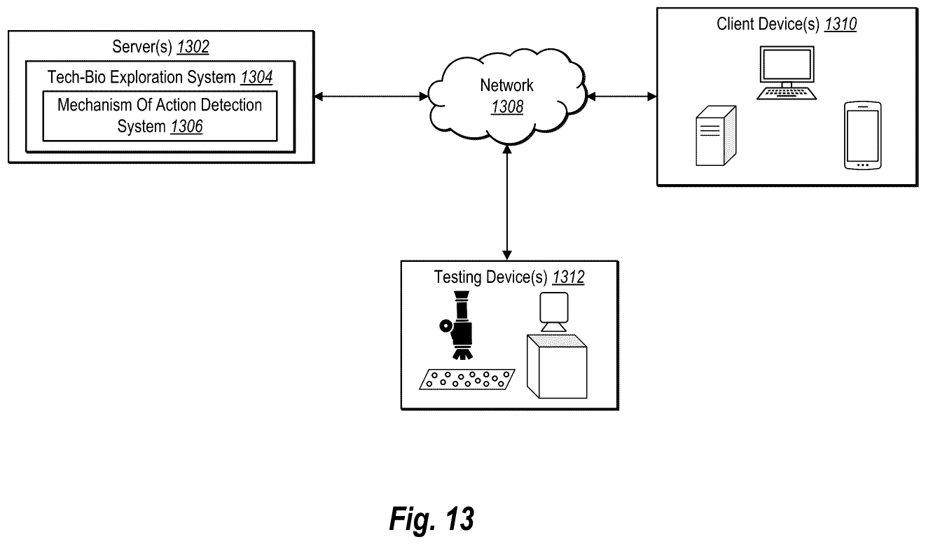

Additional detail regarding a mechanism-of-action detection system will now be provided with reference to the figures. Indeed, FIG. 1 illustrates an overview of a mechanism-of-action detection system 1306 (as shown in FIG. 13) generating MOA representations utilizing annotated digital signals from cell representations within a shared feature space that enable MOA predictions for query perturbations. For example, FIG. 1 illustrates the mechanism-of-action detection system 1306 identifying cell representation embeddings, annotating the cell representation embeddings with mechanism of actions, and generating a mechanism of action representation from annotated cell representation embeddings. Furthermore, FIG. 1 also illustrates, in some cases, the mechanism-of-action detection system 1306 determining a mechanism of action detection confidence score and predicting mechanism of actions for a mechanism of action query for a perturbation.

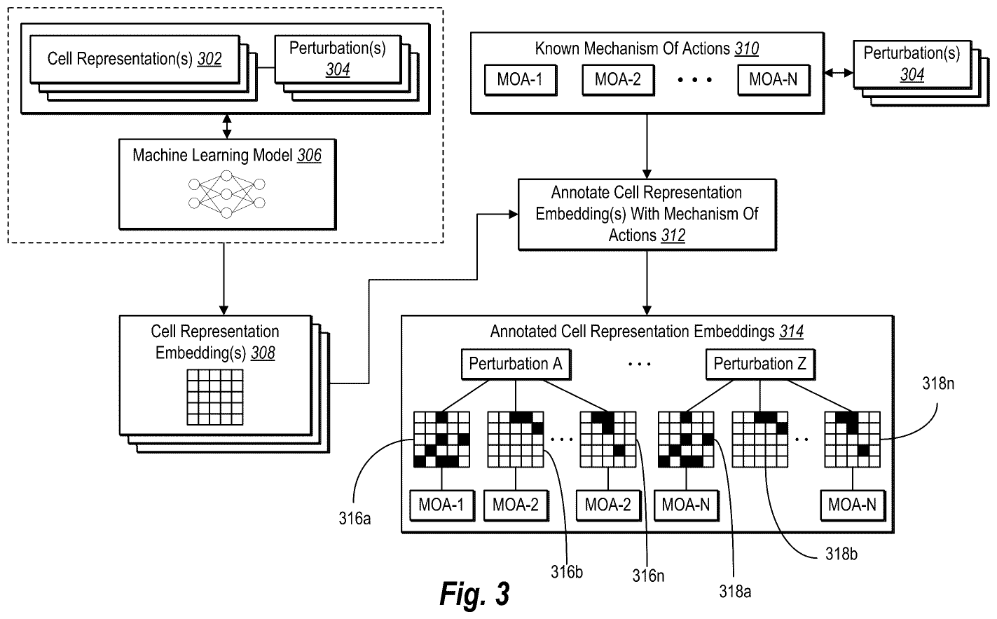

Specifically, as shown in act 102 of FIG. 1, the mechanism-of-action detection system 1306 identifies cell representation embeddings. For instance, as shown in the act 102 of FIG. 1, the mechanism-of-action detection system 1306 can access a cell data repository to identify cell representation embeddings that correspond to cell representations. In some cases, the cell representation embeddings are generated utilizing a machine learning model that is trained to predict perturbations from cell representations or generate predicted cell representations from masked cell representations. In relation to the act 102, in some cases, the mechanism-of-action detection system 1306 can generate cell representation embeddings utilizing the machine learning model. Indeed, the mechanism-of-action detection system 1306 identifying cell representation embeddings is described in greater detail below (e.g., in reference to FIG. 3).

For example, as used herein, the term “perturbation” (e.g., cell perturbation) refers to an alteration or disruption to a cell or the cell's environment (to elicit potential phenotypic changes to the cell). In particular, the term perturbation can include a gene perturbation (i.e., a gene-knockout perturbation) or a compound perturbation (e.g., a molecule perturbation or a soluble factor perturbation). These perturbations are accomplished by performing a perturbation experiment. A perturbation experiment refers to a process for applying a perturbation to a cell. A perturbation experiment also includes a process for developing/growing the perturbed cell into a resulting phenotype.

Thus, a gene perturbation can include gene-knockout perturbations (performed through a gene knockout experiment). For instance, a gene perturbation includes a gene-knockout in which a gene (or set of genes) is inactivated or suppressed in the cell (e.g., by CRISPR-Cas9 editing).

Moreover, the term “compound perturbation” can include a cell perturbation using a molecule and/or soluble factor. For instance, a compound perturbation can include reagent profiling such as applying a small molecule to a cell and/or adding soluble factors to the cell environment. Additionally, a compound perturbation can include a cell perturbation utilizing the compound or soluble factor at a specified concentration. Indeed, compound perturbations performed with differing concentrations of the same molecule/soluble factor can constitute separate compound perturbations. A soluble factor perturbation is a compound perturbation that includes modifying the extracellular environment of a cell to include or exclude one or more soluble factors. Additionally, soluble factor perturbations can include exposing cells to soluble factors for a specified duration wherein perturbations using the same soluble factors for differing durations can constitute separate compound perturbations.

Moreover, as used herein, the term “cell representation” (or “cell data”) can refer to data that indicates or represents one or more characteristics of samples or other objects (e.g., cell structure samples, chemical objects, biological objects) obtained through microscopic instruments (e.g., a microscope, gene testing device). For example, a cell representation can include a phenomic (or microscopy) image (of a perturbation). Additionally, a cell representation can include transcriptomics data that indicates molecular structures expressed in a biological (or chemical) sample (of a perturbation). For example, transcriptomics data can include an array or table of ribonucleic acid (RNA) or messenger RNA (mRNA) produced (e.g., an RNA count) in a cell or tissue sample for one or more perturbations.

Furthermore, as used herein, the term “phenomic image” (or “perturbation image”), refers to a digital image portraying a cell (e.g., a cell after applying a perturbation). For example, a phenomic image includes a digital image of a stem cell after application of a perturbation and further development of the cell. Thus, a phenomic image comprises pixels that portray a modified cell phenotype resulting from a particular cell perturbation.

As mentioned herein, the mechanism-of-action detection system 1306 can embed cell representations (e.g., phenomic images) into a low dimensional shared feature space via a generative machine learning model (e.g., a masked autoencoder model, channel-agnostic masked autoencoder model, a perturbation prediction model) to generate cell representation embeddings (e.g., perturbation image embeddings or phenomic perturbation autoencoder embeddings). As used herein, the term “cell representation embedding” (or perturbation autoencoder embeddings, phenomic perturbation autoencoder embeddings, or phenomic image embeddings) refers to a numerical representation of a cell representation (e.g., a phenomic image). For example, a cell representation embedding includes a vector representation of a cell representation generated by a machine learning model (e.g., a masked autoencoder generative model, a perturbation prediction model). Thus, a cell representation embedding includes a feature vector generated by application of various machine learning (or encoder) layers (at different resolutions/dimensionality).

In some instances, the mechanism-of-action detection system 1306 can embed other cell representations (e.g., transcriptomics representations) into a low dimensional feature space via a generative machine learning model to generate cell representation embeddings (e.g., a numerical and/or feature vector representation of transcriptomics data). For instance, a cell representation embedding can include a vector representation of transcriptomics data generated by a machine learning model.

As used herein, the term “shared feature space” (sometimes referred to “feature space” or “low dimensional feature space”) refers to a collection of features (e.g., latent features) represented utilizing a common format (or value). For instance, a shared feature space can include a framework (or mapping) that represents one or more types of data or modalities in a common format or space. In some cases, a shared feature space includes a collection of vector representations (or other values) that represent cell representation embeddings in a unified representation to enable comparisons, analysis, and/or learning across the cell representation embeddings.

As used herein, the term “machine learning model” includes a computer algorithm or a collection of computer algorithms that can be trained and/or tuned based on inputs to approximate unknown functions. For example, a machine learning model can include a computer algorithm with branches, weights, or parameters that changed based on training data to improve for a particular task. Thus, a machine learning model can utilize one or more learning techniques (e.g., supervised or unsupervised learning) to improve in accuracy and/or effectiveness. Example machine learning models include various types of decision trees, support vector machines, Bayesian networks, random forest models, or neural networks (e.g., deep neural networks, generative adversarial neural networks, convolutional neural networks, recurrent neural networks, and/or diffusion neural networks). Similarly, the term “machine learning data” refers to information, data, or files generated or utilized by a machine learning model. Machine learning data can include training data, machine learning parameters, or embeddings/predictions generated by a machine learning model.

For instance, the mechanism-of-action detection system 1306 can utilize a machine learning model to generate cell representation embeddings from cell representations. For instance, the mechanism-of-action detection system 1306 can utilize a machine learning model trained to predict perturbations from cell representations or generate predicted cell representations from masked cell representations. For example, the mechanism-of-action detection system 1306 can utilize a machine learning model to generate cell representation embeddings as described in UTILIZING MACHINE LEARNING MODELS TO SYNTHESIZE PERTURBATION DATA TO GENERATE PERTURBATION HEATMAP GRAPHICAL USER INTERFACES, U.S. patent application Ser. No. 18/526,707, filed Dec. 1, 2023 (hereinafter “US application '707”), UTILIZING COMPOUND-PROTEIN MACHINE LEARNING REPRESENTATIONS TO GENERATE BIOACTIVITY PREDICTIONS, U.S. patent application Ser. No. 18/505,728, filed Nov. 9, 2023 (hereinafter “US application '728”), UTILIZING BIOLOGICAL MACHINE LEARNING REPRESENTATIONS AND A LANGUAGE MACHINE LEARNING MODEL FOR INITIATING COMPOUND EXPLORATION PROGRAMS, U.S. patent application Ser. No. 18/521,910, filed Nov. 28, 2023 (hereinafter “US application '910”), and/or UTILIZING MACHINE LEARNING AND DIGITAL EMBEDDING PROCESSES TO GENERATE DIGITAL MAPS OF BIOLOGY AND USER INTERFACES FOR EVALUATING MAP EFFICACY, U.S. patent application Ser. No. 18/392,989, filed Dec. 21, 2023 (hereinafter “US application '989”), each of which are incorporated by reference in their entirety herein. Additionally, in some cases, the mechanism-of-action detection system 1306 can utilize a machine learning model trained to generate predicted cell representations from masked cell representations as described in UTILIZING MASKED AUTOENCODER GENERATIVE MODELS TO EXTRACT CELL REPRESENTATION AUTOENCODER EMBEDDINGS, U.S. patent application Ser. No. 18/545,399, filed Dec. 19, 2023 (hereinafter “US application '399”), which is incorporated herein by reference in its entirety.

Although the description herein sometimes refers to a singular cell or cell representation, it will be appreciated that the mechanism-of-action detection system 1306 can operate with regard to a plurality of cells (e.g., a population of cells) in relation to one or more perturbations. Thus, the mechanism-of-action detection system 1306 can apply a first perturbation to a plurality of cells, develop the plurality of cells, and capture a plurality of images. Moreover, the mechanism-of-action detection system 1306 can generate a plurality of cell representation embeddings. In some implementations, the mechanism-of-action detection system 1306 generates a cell representation embedding from a plurality of cells (e.g., by combining cell representations from a plurality of cells to form a cell embedding for a particular perturbation). Thus, for example, the mechanism-of-action detection system 1306 can generate a first cell embedding by aggregating a plurality of cell representation embeddings from a plurality of cells exposed to a first perturbation. Similarly, the mechanism-of-action detection system 1306 can generate a second cell representation embedding by aggregating a plurality of cell representation embeddings from a plurality of cells exposed to a second perturbation.

Moreover, as shown in act 104 of FIG. 1, the mechanism-of-action detection system 1306 annotates cell representation embeddings with mechanism of actions. For instance, as shown in the act 104 of FIG. 1, the mechanism-of-action detection system 1306 identifies known mechanism of actions that correspond to the cell representations of the cell representation embeddings and utilizes the known mechanism of actions to label the cell representation embeddings. Indeed, the mechanism-of-action detection system 1306 annotating the cell representation embeddings with mechanism of actions is described in greater detail below (e.g., in reference to FIG. 3).

As used herein, the term “mechanism of action” refers to a (data representation of) biochemical process or interaction (or a data representation thereof). In particular, a mechanism of action can include a biochemical process or interaction through which a perturbation (e.g., a compound perturbation) is accomplished (or exerted) within a biological system. For example, a mechanism of action can represent a particular interaction with a specific component of a biological system (e.g., receptors, enzymes, ion channels, molecular targets, cell targets, tissue targets) to achieve a desired perturbation effect (e.g., a compound's therapeutic effect). As an example, a mechanism of action can include biochemical processes and/or interactions, such as, but not limited to, aurora kinase inhibitors, histone deacetylase inhibitors, heat shock protein inhibitor, receptor agonists, gene expression modulators, cell membrane disruptors, neurotransmitter modulators, and/or mechanistic target of rapamycin (mTOR) inhibitors.

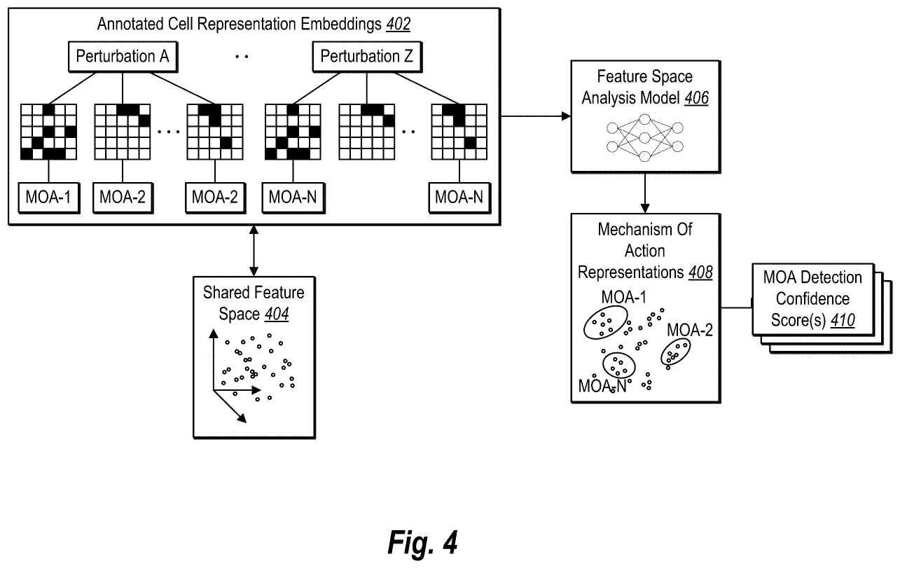

Additionally, as shown in act 106 of FIG. 1, the mechanism-of-action detection system 1306 generates a mechanism of action representation from annotated cell representation embeddings. In particular, as shown in the act 106 of FIG. 1, the mechanism-of-action detection system 1306 analyzes annotated cell representation embeddings within a shared feature space (of the embeddings) to define (or determine) clusters of cell representation embeddings that correspond to a mechanism of action. Indeed, as shown in the act 106 of FIG. 1, the mechanism-of-action detection system 1306 isolates (or identifies) clusters of cell representation embeddings that represent one or more mechanism of actions (e.g., through mechanism of action annotations). For instance, the mechanism-of-action detection system 1306 generates a mechanism of action representation that represents data signals from the embeddings that relate to (or correspond to) a mechanism of action from the clusters (or cluster features) of the cell representation embeddings annotated with one or more mechanism of actions. Indeed, the mechanism-of-action detection system 1306 generating a mechanism of action representation is described in greater detail below (e.g., in reference to FIG. 4).

As used herein, the term “mechanism of action representation” refers to a collection of digital signals that represent or correspond to a mechanism of action. In particular, a mechanism of action representation can indicate relationships between cell representation signals (e.g., via cell representation embeddings) and a particular mechanism of action. For instance, a mechanism of action representation can include a cluster (or a representation or feature derived from a cluster) of cell representation embeddings of a shared feature space that correspond to a particular mechanism of action (e.g., via annotations of a known mechanism of action as described herein). In some cases, the mechanism of action representation can include a feature or characteristic of a cluster of annotated cell representation embeddings (for the particular mechanism of action).

Furthermore, as used herein, the term “embedding cluster” refers to a grouping of data points (e.g., cell representation embeddings) within a shared feature space. Indeed, an embedding cluster can include a grouping of cell representation embeddings that are near in distance within a shared feature space (e.g., in a determined proximity) to indicate similarities or relatedness of the cell representation embeddings. For example, the mechanism-of-action detection system 1306 can generate cell representation embedding utilizing various clustering algorithms, such as, but not limited to, k-means clustering, hierarchical clustering, and/or density based spatial clustering. In addition, a feature or characteristic of an embedding cluster can include, but is not limited to, a cluster centroid and/or a cluster mean.

In one or more instances, the mechanism-of-action detection system 1306 utilizes similarity measures to generate cell representation embedding clusters (for the mechanism of action representations). For instance, the mechanism-of-action detection system 1306 can utilize a similarity measure that quantifies similarities and/or dissimilarities between embeddings in a shared feature space. For instance, the mechanism-of-action detection system 1306 an utilize a cosine similarity and/or Euclidean distance between cell representation embeddings in a shared feature space.

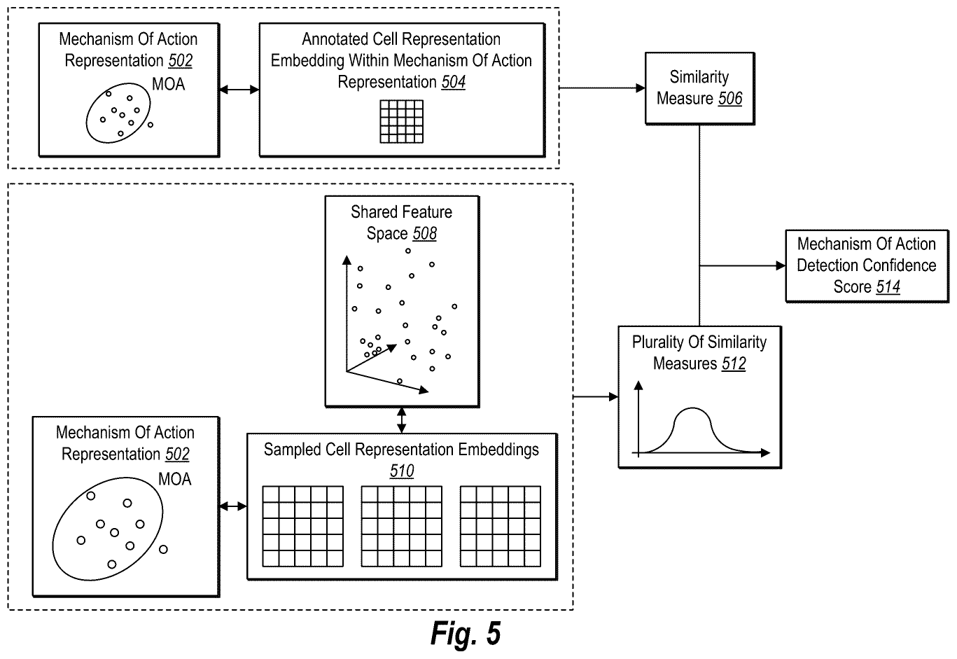

In some cases, as shown in act 108 of FIG. 1, the mechanism-of-action detection system 1306 can determine a mechanism of action detection confidence score. As shown in the act 108, in some instances, the mechanism-of-action detection system 1306 generates a mechanism of action detection confidence score that indicates whether the mechanism of action representation (and the machine learning model generating the embeddings) provides a meaningful signal for deducing the corresponding mechanism of action from cell representation signals represented in the cell representation embeddings. In particular, the mechanism-of-action detection system 1306 determines a similarity measure between one or more cell representation embeddings within the mechanism of action representation and the mechanism of action representation (e.g., a representation of a cluster feature). Moreover, the mechanism-of-action detection system 1306 determines a plurality of similarity measures (e.g., a distribution of the similarity measures) between the mechanism of action representation and sampled cell representation embeddings outside of the mechanism of action representation. In one or more embodiments, the mechanism-of-action detection system 1306 compares the similarity measure with the plurality of similarity measures to determine the mechanism of action detection confidence score. Indeed, the mechanism-of-action detection system 1306 can determine a mechanism of action detection confidence score as described in greater detail below (e.g., in reference to FIG. 5).

As used herein, the term “mechanism of action detection confidence score” refers to a value (or score) that represents whether a mechanism of action representation (and the machine learning model that generates the cell representation embeddings) provides a meaningful signal for deducing a corresponding mechanism of action from cell representation signals represented in cell representation embeddings. In particular, a mechanism of action detection confidence score can include a value or a score determined from comparing a similarity measure between one or more cell representation embeddings within the mechanism of action representation and the mechanism of action representation and a plurality of similarity measures between the mechanism of action representation and sampled cell representation embeddings outside of the mechanism of action representation. For instance, the mechanism of action detection confidence score can include a score (e.g., a z-score) or value (e.g., 0 to 1, 0 to 10) that indicates a deviation (e.g., a standard deviation, mean absolute deviation) between the above mentioned compared similarity measure and the plurality of similarity measures.

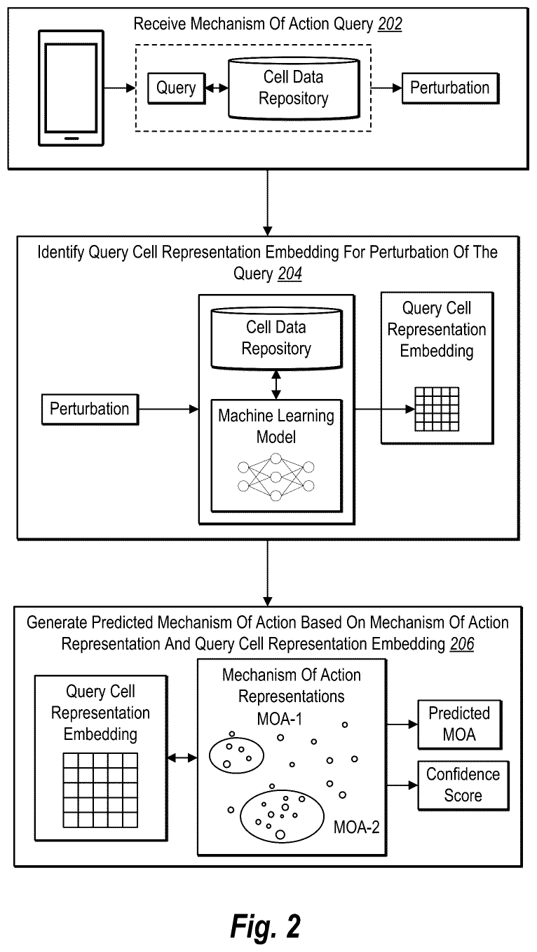

In some implementations, as shown in act 110 of FIG. 1, the mechanism-of-action detection system 1306 can predict a mechanism of action for a mechanism of action query for a perturbation. For instance, as shown in the act 110, the mechanism-of-action detection system 1306 can utilize a query (e.g., a cell representation embedding representing the query or a perturbation designated in the query) with the mechanism of action representations to identify a predicted mechanism of action. In particular, the mechanism-of-action detection system 1306 can determine a mechanism of action representation within a shared feature space that is similar to the query cell representation embedding to generate a mechanism of action corresponding to the mechanism of action representation as the predicted mechanism of action. In addition, as shown in the act 110, the mechanism-of-action detection system 1306 an also generate a confidence score for the predicted mechanism of action in relation to the query. Indeed, the mechanism-of-action detection system 1306 can predict a mechanism of action for a mechanism of action query for a perturbation as described in greater detail below (e.g., in reference to FIGS. 2, 6, and 7).

For example, FIG. 2 illustrates an overview of the mechanism-of-action detection system 1306 predict a mechanism of action for a mechanism of action query (for a perturbation). Indeed, FIG. 2 illustrates the mechanism-of-action detection system 1306 receiving a mechanism of action query, identifying a query cell representation embedding for a perturbation of the query, and generating a predicted mechanism of action based on a mechanism of action representation and query cell representation embedding (corresponding to the mechanism of action query).

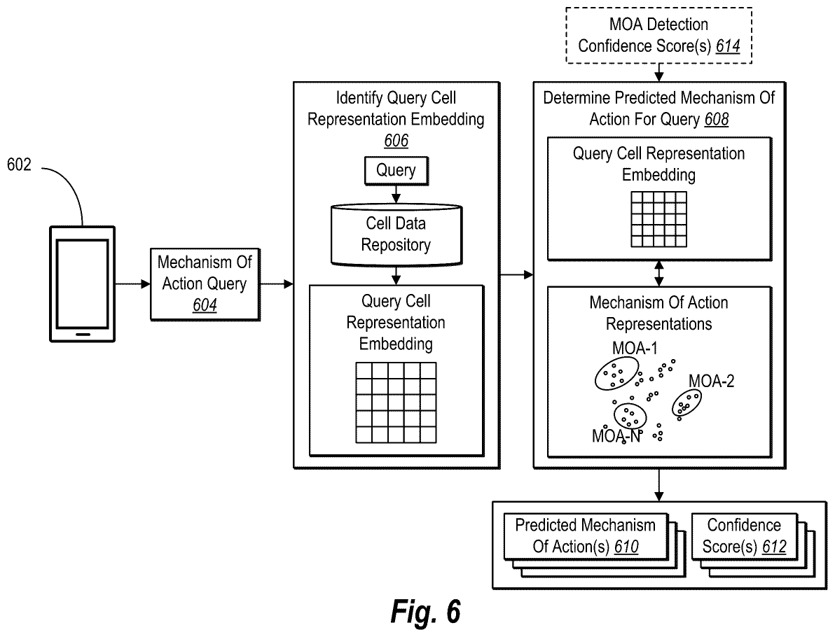

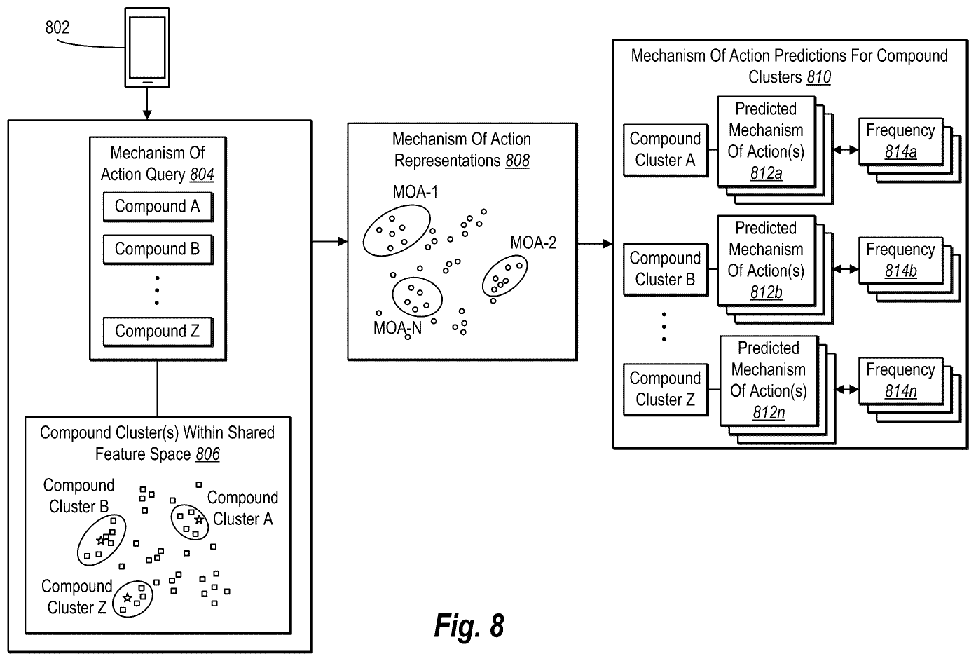

In particular, as shown in act 202 of FIG. 2, the mechanism-of-action detection system 1306 receives a mechanism of action query. In particular, the mechanism-of-action detection system 1306 can receive a mechanism of action query for a perturbation that represents a request to generate (or predict) mechanism of actions that correspond to the perturbation. As shown in the act 202, the mechanism-of-action detection system 1306 can identify a particular perturbation (from a cell data repository) that represents the query. Indeed, the mechanism-of-action detection system 1306 receiving a mechanism of action query is described in greater detail below (e.g., in reference to FIGS. 6 and 8).

As used herein, the term “mechanism of action query” refers to a prompt or selection of a perturbation (or cell data) to request an MOA detection analysis of the perturbation (or cell data). For example, a mechanism of action query can include a selection of a perturbation from a dataset of perturbations to initiate (or cause) the mechanism-of-action detection system 1306 to generate predicted MOAs for the perturbation (from cell representation embeddings related to the perturbation). In one or more instances, a mechanism of action query can include, but is not limited to, a dropdown menu list selection of a perturbation and/or a text input indicating a command for an MOA detection of a particular perturbation. In some cases, a mechanism of action query can include a selected or provided list of compounds for a request to detect MOAs related (or predicted) for the list of compounds.

Furthermore, as shown in act 204 of FIG. 2, the mechanism-of-action detection system 1306 identifies a query cell representation embedding for a perturbation of the query. For instance, as shown in the act 204, the mechanism-of-action detection system 1306 utilizes the perturbation (corresponding to the mechanism of action query) with a cell data repository to identify a cell representation embedding for the query as a query cell representation embedding. Indeed, in some cases, the mechanism-of-action detection system 1306 identifies a cell representation embedding that is generated by a machine learning model (as described above) from a cell representation that depicts (or corresponds) to the perturbation. Indeed, the mechanism-of-action detection system 1306 identifying a query cell representation embedding for a perturbation of a query is described in greater detail below (e.g., in reference to FIG. 2).

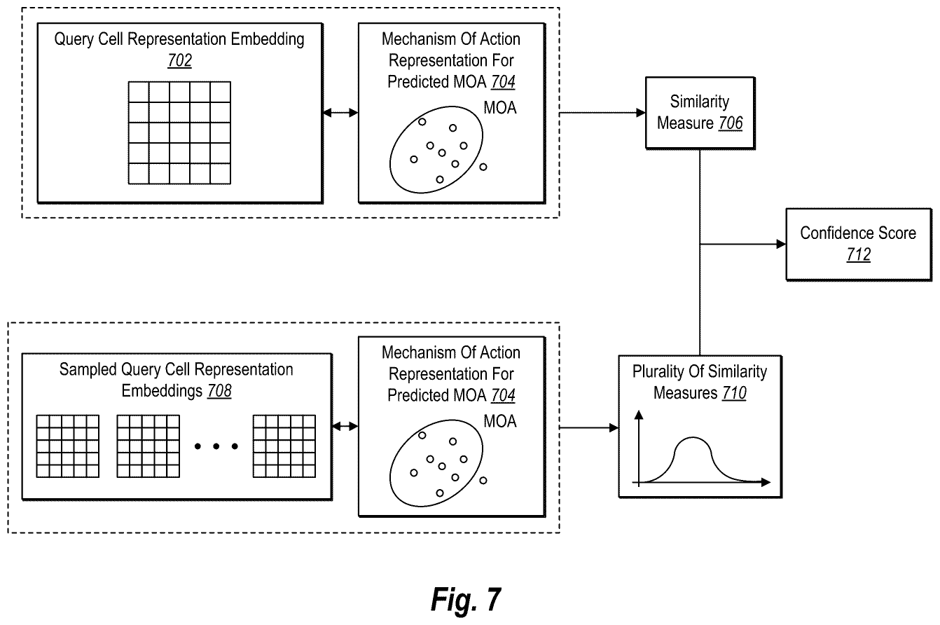

Additionally, as shown in act 206 of FIG. 2, the mechanism-of-action detection system 1306 generates a predicted mechanism of action based on a mechanism of action representation and query cell representation embedding. For instance, as shown in the act 206 of FIG. 2, the mechanism-of-action detection system 1306 compares the query cell representation embedding to one or more mechanism of action representations within a shared feature space to identify a predicted mechanism of action for the query. In particular, the mechanism-of-action detection system 1306 utilizes similarity measures between the query cell representation embedding to one or more mechanism of action representations to select a mechanism of action representation. Moreover, the mechanism-of-action detection system 1306 utilizes a mechanism of action corresponding to the mechanism of action representation as the predicted mechanism of action for the query. As further shown in the act 206, the mechanism-of-action detection system 1306 also generates a confidence score for the predicted MOA. Indeed, the mechanism-of-action detection system 1306 generating a predicted MOA and a confidence score is described in greater detail below (e.g., in reference to FIGS. 6 and 7).

As used herein, the term “prediction confidence score” (sometimes referred to as “confidence score”) refers to a value or score that indicates a measure of similarities (or likeness) between a mechanism of action representation and a query cell representation embedding within a shared feature space. In some cases, the prediction confidence score can include a value or score that indicates whether a query cell representation embedding exhibits one or more meaningful signals of a mechanism of action representation in comparison to the other sampled query cell representation embeddings. For instance, the prediction confidence score can include a score (e.g., a z-score) or value (e.g., 0 to 1, 0 to 10) that indicates a similarity measure between a mechanism of action representation and a query cell representation embedding or a deviation (e.g., a standard deviation, mean absolute deviation) between a similarity measure (of the mechanism of action representation and the query cell representation embedding) and a plurality of similarity measures (of the mechanism of action representation and other sampled query cell representation embeddings).

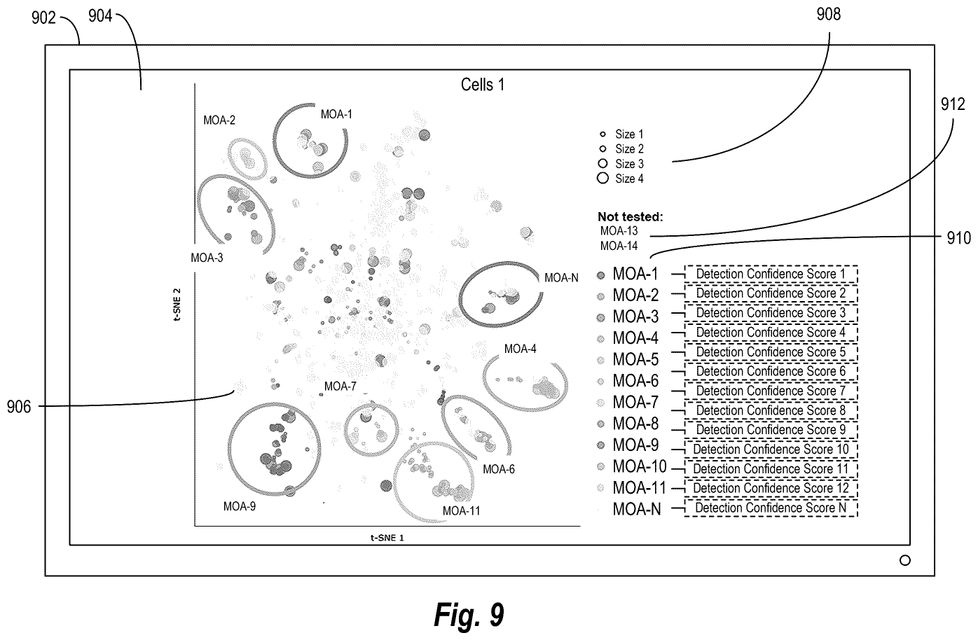

In some cases, the mechanism-of-action detection system 1306 further utilizes predicted MOAs and/or MOA representations to display one or more graphical user interfaces that indicate a mechanism of action representation (and detection confidence scores). Furthermore, the mechanism-of-action detection system 1306 can also display one or more graphical user interfaces to display a predicted MOA and/or confidence scores related to the predicted MOA. Additionally, the mechanism-of-action detection system 1306 can also display selectable options to receive a selection of multiple compounds and display generated MOA predictions for the selected compounds (in accordance with one or more implementations herein). Indeed, the mechanism-of-action detection system 1306 can display various user interfaces for mechanism of action representations, detection confidence scores, predicted mechanism of actions in response to mechanism of action queries, and/or confidence scores as described in greater detail below (e.g., in reference to FIGS. 9, 10, and 11).

As mentioned above, although conventional systems can utilize computer-based models to extract and analyze digital signals for images portraying cells, these conventional systems often have a number of technical shortcomings with regard to efficiency, flexibility, and accuracy. In particular, many conventional systems cannot easily and efficiently draw accurate digital deductions (or predictions) of certain biological relationships from cell data (e.g., perturbations represented in microscopy images).

For example, in many cases, conventional systems often rely on user observation and annotation of cell data to draw biological relationship inference observations from the cell data. In many cases, due to the vast number digital signals from cell data, it is often difficult and inefficient to observe or draw biological relationship inferences from the cell data. For example, in many instances, due to the substantial number of features and signals available within cell data and the imperceptibility of some biological relationships within cell data, utilizing computing devices to drawing conclusions from gathered cell data requires extensive user navigation, data manipulation, time, and computing resources. Such approaches are often inefficient.

Moreover, often, conventional systems utilize models that rely on formulaic statistical approaches to estimate or infer some types of biological relationships from gathered cell data. However, such conventional systems are often inaccurate and rigid. For example, many conventional systems are unable to capture or determine nuanced inferences from cell data via the digital signals of the cell data using formulaic statistical approaches. These approaches, oftentimes lead to inaccurate inferences and, in many cases, conventional systems are unable to accurately identify a targeted biological relationship from cell data without specifically training a model framework to identify the targeted biological relationship (via computationally expensive and time extensive training approaches).

Indeed, in some cases, conventional systems are able to draw inferences from cell data when a model is trained specifically for the inference. Such models trained by conventional systems are unable to scale to deduce other types of inferences not exposed to (or trained on) the model. Indeed, in many instances, model trained by conventional systems are unable to accurately draw additional biological relationship inferences from a model trained specifically for a single type of biological relationship inference. Accordingly, in many cases, conventional systems rigidly and inefficiently train model frameworks to draw specific types of biological relationship inferences from cell data.

As suggested by the foregoing, the mechanism-of-action detection system 1306 provides a variety of technical advantages relative to conventional systems. Unlike conventional systems, the mechanism-of-action detection system 1306 can utilize digital signals from cell representations and known mechanism of action relationships to efficiently generate mechanism of action representations and utilize the mechanism of action representations to generate accurate mechanism of action predictions from cell data. Indeed, the mechanism-of-action detection system 1306 can automatically generate the mechanism of action representations from known mechanism of action relationships with existing cell representation embeddings by automatically annotating the cell representation embeddings using the known mechanism of action relationships to highlight the mechanism of action relationships in a shared feature space (e.g., via clustering). Accordingly, unlike many conventional systems, the mechanism-of-action detection system 1306 can efficiently generate mechanism of action representations that are useable for mechanism of action detections in other cell data without extensive user navigation, data manipulation, time, and computing resources for training models.

Furthermore, the mechanism-of-action detection system 1306 also improves accuracy and flexibility of deducing biological relationship inferences from cell data. In particular, in contrast to many conventional systems that rely on formulaic statistical approaches, the mechanism-of-action detection system 1306 can consider a dynamic number of digital signals corresponding to cell representation embeddings in a mechanism of action representation (generated in accordance with one or more implementations herein) to flexibly draw accurate deductions of MOAs from cell data (e.g., perturbation queries). For instance, unlike many conventional systems, the mechanism-of-action detection system 1306 can flexibly utilize cell representation embeddings generated from machine learning models trained for various tasks (e.g., perturbation predictions, reconstruction of masked cell representations) to accurately deduce MOAs from cell data. Indeed, the mechanism-of-action detection system 1306 can efficiently and flexibly utilize the machine learning models trained for various tasks to generate the MOA representations and deduce MOA predictions from cell data without targeted training for the MOA task.

Moreover, the mechanism-of-action detection system 1306 can accurately detect MOAs from cell data. In particular, the mechanism-of-action detection system 1306 can utilize MOA representations generated from annotated cell representation embeddings to accurately identify relationships between cell data and imperceptible mechanism of actions. In addition, the mechanism-of-action detection system 1306 can also generate mechanism of action detection confidence scores for MOA representations to provide a measurement of the detectability of a mechanism of action (from a mechanism of action representation) to determine a reliability of a mechanism of action representation. Furthermore, the mechanism-of-action detection system 1306 can also generate a prediction confidence score that specifically determines a measure of confidence between a particular mechanism of action query (e.g., a query perturbation) against a detected MOA representation for the mechanism of action query. Indeed, in many instances, the utilization of the mechanism of action detection confidence score and the prediction confidence score improves the accuracy of MOA detection from perturbations and other cell data.

As mentioned above, the mechanism-of-action detection system 1306 can identify cell representation embeddings and annotate the cell representation embeddings with mechanism of actions (MOAs) that correspond to the cell representation embeddings. For example, FIG. 3 illustrates the mechanism-of-action detection system 1306 identifying cell representation embeddings. In addition, FIG. 3 also illustrates the mechanism-of-action detection system 1306 annotating the identified cell representation embeddings with MOAs.

In particular, as shown in FIG. 3, the mechanism-of-action detection system 1306 can access cell representation(s) 302 that correspond to perturbation(s) 304. Indeed, in some instances, the mechanism-of-action detection system 1306 can utilize the cell representation(s) 302 with a machine learning model 306 to generate the cell representation embedding(s) 308. Indeed, in one or more implementations, the mechanism-of-action detection system 1306 accesses a cell data repository (of a tech-bio exploration system 1304) that includes cell representations, (tagged and/or predicted) perturbations for the cell representations, and cell representation embeddings generated for the cell representations (as described above). For instance, the mechanism-of-action detection system 1306 can access the cell data repository to identify (or access) the cell representation embedding(s) 308 for the cell representation(s) 302.

In addition, as shown in FIG. 3, the mechanism-of-action detection system 1306 also identifies known mechanism of actions 310 for the perturbation(s) 304. Indeed, in one or more instances, the perturbation(s) 304 include metadata (or tags) that indicate one or more mechanism of actions known to correspond with a particular perturbation. As an example, the mechanism-of-action detection system 1306 can identify a compound (as the perturbation(s) 304) and known mechanism of actions for the compound. The mechanism-of-action detection system 1306 can utilize the known mechanism of actions 310 corresponding to the perturbation(s) 304 to annotate cell representation embeddings corresponding to the perturbation(s) 304.

For instance, as shown in act 312 of FIG. 3, the mechanism-of-action detection system 1306 annotates cell representations with mechanism of actions (from the known mechanism of actions 310). In particular, the mechanism-of-action detection system 1306 utilizes annotates (or tags) a cell representation embedding from the cell representation embedding(s) 308 with a known mechanism of action from the known mechanism of actions 310 that correspond to the same perturbation from the perturbation(s) 304.

Indeed, as shown in FIG. 3, the mechanism-of-action detection system 1306 generates annotated cell representation embeddings 314 that include cell representation embeddings annotated with one or more known mechanism of actions. For instance, as shown in FIG. 3, the mechanism-of-action detection system 1306 annotates the cell representation embeddings 316a, 316b, 316n corresponding to the perturbation 1 with an MOA 1 and/or an MOA-2. Indeed, the mechanism-of-action detection system 1306 can annotate an embedding for a perturbation with different MOAs (if there are multiple known MOAs for a particular perturbation). Additionally, as shown in FIG. 3, the mechanism-of-action detection system 1306 annotates the cell representation embeddings 318a, 318n corresponding to the perturbation N with an MOA-N. In addition, as shown in FIG. 3, mechanism-of-action detection system 1306 foregoes tagging the cell representation embedding 318b corresponding to the perturbation N with an MOA. Indeed, although FIG. 3 illustrates a specific annotation of cell representation embeddings, the mechanism-of-action detection system 1306 can annotate various numbers of cell representation embeddings with a variety of MOAs (e.g., the same MOA, multiple MOAs, different MOAs for different types of cell representation embeddings).

In some implementations, the mechanism-of-action detection system 1306 can apply an annotation to one or more cell representation embeddings without knowing the precise mechanism of action. For example, the mechanism-of-action detection system 1306 can identify a novel or new cluster of embeddings that are not associated with a previously known mechanism of action. The mechanism-of-action detection system 1306 can utilize this grouping or cluster of embeddings as a “new MOA” or “novel MOA” and apply a corresponding annotation to the corresponding cell representation embeddings. Moreover, the mechanism-of-action detection system 1306 can generate a mechanism of action representation for the new MOA (i.e., previously unknown MOA), determine a detection confidence score for the new MOA, and/or generate MOA predictions corresponding to the new MOA for future queries.

In one or more instances, the mechanism-of-action detection system 1306 utilizes existing cell representation embeddings from a cell data repository of a tech-bio exploration system 1304. In particular, the tech-bio exploration system 1304 can utilize one or more machine learning models to generate and store cell representation embeddings from cell data (e.g., phenomic images and/or transcriptomics data). Indeed, the tech-bio exploration system 1304 can utilize a machine learning model that predicts perturbations from phenomic images and/or reconstructs phenomic images from masked phenomic images as described above.

Furthermore, the mechanism-of-action detection system 1306 can identify known mechanism of actions that correspond to cell representations (e.g., cell representations representing perturbations). Moreover, the mechanism-of-action detection system 1306 can label or annotate the cell representation embeddings from the identified cell representations that correspond to the known mechanism of actions. In some cases, the mechanism-of-action detection system 1306 utilizes tags and/or metadata corresponding to the cell representation embeddings to annotate the cell representation embeddings with the identified known mechanism of actions.

Although one or more instances illustrate a one-to-one annotation of a mechanism of action to a cell representation embedding, the mechanism-of-action detection system 1306 can annotate a cell representation embedding with a plurality of mechanism of actions that correspond to the cell representation embedding.

In some cases, a cell representation embedding can also include an embedding generated from multiple cell representations or perturbations (e.g., an aggregated cell representation embedding). Indeed, the mechanism-of-action detection system 1306 can annotate an aggregated cell representation embedding with one or more mechanism of actions that correspond to the cell representations or perturbations represented in the aggregated cell representation embedding.

As mentioned above, the mechanism-of-action detection system 1306 can generate a mechanism of action representation from annotated cell representation embeddings. For instance, FIG. 4 illustrates the mechanism-of-action detection system 1306 generating mechanism of action representations from annotated cell representation embeddings. In particular, FIG. 4 illustrates the mechanism-of-action detection system 1306 generating mechanism of action representations that represent (or indicate) cell representation digital signals (via cell representation embeddings) that are indicative of (or signifies) a particular MOA.

For instance, as shown in FIG. 4, the mechanism-of-action detection system 1306 utilizes annotated cell representation embeddings 402 (as described above) with a shared feature space 404 to generate the mechanism of action representations 408. In particular, as shown in FIG. 4, the mechanism-of-action detection system 1306 utilizes a feature space analysis model 406 to analyze the annotated cell representation embeddings 402 within the shared feature space 404 (that includes the annotated cell representation embeddings 402 and other cell representation embeddings) to generate embedding clusters that cluster similar cell representation embeddings in the shared feature space 404.

Moreover, the mechanism-of-action detection system 1306 identifies (or isolates) embedding clusters that include the annotated cell representation embeddings to generate an MOA representation. For instance, as shown in FIG. 4, the mechanism of action representations 408, generated by the mechanism-of-action detection system 1306, include separate embedding clusters that are indicative of MOAs (e.g., MOA-1, MOA-2, MOA-N). Indeed, the mechanism-of-action detection system 1306 can generate an embedding cluster (e.g., the cluster corresponding to MOA-1 in the mechanism of action representations 408) from annotated cell representation embeddings annotated with MOA-1 as an MOA representation for the MOA-1 within the shared feature space 404.

In addition, as shown in FIG. 4, the mechanism-of-action detection system 1306 can determine MOA detection confidence score(s) 410 for the MOAs represented within the mechanism of action representations 408. As an example, the mechanism-of-action detection system 1306 can generate a MOA detection confidence score for the embedding cluster of MOA-1 (i.e., an MOA representation for MOA-1). Moreover, the mechanism-of-action detection system 1306 can generate an additional MOA detection confidence score for the embedding cluster of MOA-2 (i.e., an MOA representation for MOA-2). Indeed, the mechanism-of-action detection system 1306 can generate MOA detection confidence scores as described in greater detail below (e.g., in reference to FIG. 5).

In one or more instances, the mechanism-of-action detection system 1306 can utilize a clustering algorithm as the feature space analysis model to generate cell representation embedding clusters within a shared feature space. For instance, the mechanism-of-action detection system 1306 can utilize a clustering algorithm to determine similarity measures between cell representation embeddings (e.g., including annotated cell representation embeddings) within the shared feature space. Indeed, the mechanism-of-action detection system 1306 can utilize the clustering algorithm to cluster similar cell representation embeddings within the shared feature space. Indeed, the mechanism-of-action detection system 1306 can utilize a variety of clustering algorithms, such as, but not limited to k-means clustering, hierarchical clustering, and/or density based spatial clustering to generate one or more embedding clusters (of the MOA representations).

In addition, the mechanism-of-action detection system 1306 can utilize a variety of similarity measures to cluster the one or more cell representation embeddings within the shared feature space. In some cases, the mechanism-of-action detection system 1306 utilizes a feature space distance measure between embeddings as the similarity measure. For instance, the mechanism-of-action detection system 1306 can utilize cosine similarities and/or Euclidian distances between one or more cell representation embeddings within the shared feature space.

Furthermore, in one or more instances, an MOA representation can include a cluster of one or more annotated cell representation embeddings in a shared feature space. For example, the MOA representation can include a representation of a cluster of one or more annotated cell representation embeddings to define an approximation or approximation of the annotated cell representations embeddings that represent a particular MOA. For instance, in some cases, the mechanism-of-action detection system 1306 can determine a centroid from a cluster of one or more annotated cell representation embeddings as the MOA representation. Indeed, the MOA representation can represent a grouping of cell representation embedding data signals that exhibit features that correspond to one or more particular annotated MOAs.

As also mentioned above, the mechanism-of-action detection system 1306 can determine an MOA detection confidence score for an MOA representation. In particular, the mechanism-of-action detection system 1306 can determine an MOA detection confidence score that indicates whether annotated cell representation embeddings (generated by a machine learning model) within an MOA representation provide a meaningful signal for deducing the particular MOA in comparison to machine learning generated cell representation embeddings outside of the MOA representation. Indeed, FIG. 5 illustrates the mechanism-of-action detection system 1306 generating an MOA detection confidence score for an MOA representation.

For example, as shown in FIG. 5, the mechanism-of-action detection system 1306 compares a mechanism of action representation 502 with an annotated cell representation embedding 504 (within the mechanism of action representation) to generate a similarity measure 506. In particular, in some cases, the mechanism-of-action detection system 1306 compares a cluster feature of the mechanism of action representation 502 (e.g., a cluster centroid) to the annotated cell representation embedding 504 to generate the similarity measure 506. Indeed, the similarity measure 506 can indicate a measure of the similarities (or likeness) between the mechanism of action representation 502 and the annotated cell representation embedding 504 within a shared feature space. Although one or more embodiments illustrate the mechanism-of-action detection system 1306 comparing a singular annotated cell representation embedding, the mechanism-of-action detection system 1306 can compare multiple annotated cell representation embeddings (or an aggregated annotated cell representation embedding) to a MOA representation to generate the similarity measure.

In addition, as shown in FIG. 5, the mechanism-of-action detection system 1306 also samples cell representation embeddings (e.g., sampled cell representation embeddings 510) from a shared feature space 508. For example, the mechanism-of-action detection system 1306 can sample the sampled cell representation embeddings 510 as embeddings that are outside (of an embedding cluster) of the mechanism of action representation 502. In some cases, the mechanism-of-action detection system 1306 randomly samples the shared feature space 508 outside of the mechanism of action representation 502 to determine the sampled cell representation embeddings 510. Moreover, as shown in FIG. 5, the mechanism-of-action detection system 1306 compares the sampled cell representation embeddings 510 to the mechanism of action representation 502 (e.g., a cluster centroid) to generate a plurality of similarity measures 512.

Indeed, the plurality of similarity measures 512 can include a sample (or example) of how cell representation embeddings in a shared feature space relate to the mechanism of action representation 502 (e.g., to determine if the mechanism of action representation 502 provides a meaningful signal for deducing a particular MOA). In some cases, the plurality of similarity measures 512 are utilized as an empirical null distribution of similarity measure scores for the particular MOA. Indeed, the mechanism-of-action detection system 1306 can utilize a plurality of similarity measures 512 that includes a distribution of similarity measures between the MOA representation and sampled cell representation embeddings from the shared feature space to compare the MOA representation's ability to distinguish between embedding signals within the MOA representation and outside of the MOA representation to deduce the particular MOA.

For instance, as shown in FIG. 5, the mechanism-of-action detection system 1306 compares the similarity measure 506 to the plurality of similarity measures 512 to generate a mechanism of action detection confidence score 514. As mentioned above, the mechanism-of-action detection system 1306 compares the similarity measure 506 to the plurality of similarity measures 512 to determine if the annotated cell representation embeddings within the MOA representation 502 provide a meaningful signal for deducing a particular MOA in comparison to embeddings outside of the MOA representation 502. Indeed, the mechanism-of-action detection system 1306 can generate a comparative value as the mechanism of action detection confidence score based on the comparison between the similarity measure 506 and the plurality of similarity measures 512.

As mentioned above, the mechanism-of-action detection system 1306 can utilize a variety of similarity measures to compare cell representation embeddings (and/or mechanism of action representations) within a shared feature space. In some cases, the mechanism-of-action detection system 1306 utilizes a feature space distance measure between embeddings as the similarity measure. For instance, the mechanism-of-action detection system 1306 can utilize cosine similarities and/or Euclidian distances between one or more cell representation embeddings (and/or mechanism of action representations) within the shared feature space (to determine a mechanism of action detection confidence score).

In some instances, the mechanism-of-action detection system 1306 can generate a plurality of similarity measures as a distribution of similarity measures between a particular MOA representation and sampled cell representation embeddings in the shared feature space. For instance, the mechanism-of-action detection system 1306 can generate a distribution of similarity measures that represents how a sampling of cell representation embeddings compare to the particular MOA representation as a benchmark for identifying meaningful and non-meaningful MOA representations (in comparison to similarity measures of annotated cell representation embeddings within the MOA representation).

For instance, if one or more annotated cell representation embeddings have a similarity measure that is indifferent from a distribution of similarity measures for the MOA representation, the mechanism-of-action detection system 1306 can determine that the MOA representation includes data signals in the form of annotated cell representation embeddings that are not meaningful in detection of the particular MOA. Moreover, if one or more annotated cell representation embeddings have a similarity measure that is different from a distribution of similarity measures for the MOA representation, the mechanism-of-action detection system 1306 can determine that the MOA representation includes data signals in the form of annotated cell representation embeddings that are meaningful in detection of the particular MOA.

Furthermore, the mechanism-of-action detection system 1306 can utilize a variety of measurement types to compare a similarity measure of the one or more annotated cell representation embeddings to a distribution of similarity measures for the MOA representation. For instance, the mechanism-of-action detection system 1306 can determine a standard deviation between the similarity measure of the one or more annotated cell representation embeddings to a distribution of similarity measures for the MOA representation. In some cases, the mechanism-of-action detection system 1306 can also determine a mean absolute deviation between the similarity measure of the one or more annotated cell representation embeddings to a distribution of similarity measures for the MOA representation. Furthermore, the mechanism-of-action detection system 1306 can determine whether the comparison value (e.g., the standard deviation, mean absolute deviation) satisfies a meaningful detection threshold (e.g., a threshold value representing a deviation value that indicates a meaningful signal).

In some instances, the mechanism-of-action detection system 1306 utilizes the comparison value (e.g., the standard deviation, mean absolute deviation) to determine a MOA detection confidence score. For example, the mechanism-of-action detection system 1306 can determine MOA detection confidence score by utilizing the comparison value (e.g., the standard deviation, mean absolute deviation). In some cases, the mechanism-of-action detection system 1306 can increase a MOA detection confidence score as the comparison value (e.g., the standard deviation, mean absolute deviation) increases (e.g., indicating a greater difference between the similarity measure of the one or more annotated cell representation embeddings to a distribution of similarity measures for the MOA representation).

In addition, the mechanism-of-action detection system 1306 can store generated MOA representations (e.g., within a tech-bio exploration system 1304). Moreover, the mechanism-of-action detection system 1306 can also store generated MOA detection confidence scores for MOA representations (e.g., within the tech-bio exploration system 1304). In some instances, the mechanism-of-action detection system 1306 stores the generated MOA representations and/or the MOA detection confidence scores to utilize the generated MOA representations and/or the MOA detection confidence scores with a variety of tools (e.g., tech-bio exploration tools) of the tech-bio exploration system 1304. Additionally, in one or more instances, the mechanism-of-action detection system 1306 can store one or more distributions of similarity measures (for comparisons with sampled cell representation embeddings outside of the MOA representations) of one or more MOA representations (generated in accordance with one or more implementations herein). Indeed, the mechanism-of-action detection system 1306 can also store one or more MOA detection confidence scores generated for the one or more MOA representations.

As mentioned above, the mechanism-of-action detection system 1306 can utilize MOA representations, within the shared feature space, to predict MOAs for a query cell representation (of a perturbation). For instance, FIG. 6 illustrates the mechanism-of-action detection system 1306 predicting a mechanism of action for a query (corresponding to a perturbation). In particular, FIG. 6 illustrates the mechanism-of-action detection system 1306 receiving a mechanism of action query, identifying a query cell representation embedding for the query, and utilizing the query cell representation embedding with one or more mechanism of action representations to generate a predicted mechanism of action.

In particular, as shown in FIG. 6, the mechanism-of-action detection system 1306 receives a mechanism of action query 604 from a computing device 602 (e.g., a client device). In one or more instances, the mechanism of action query 604 can correspond to (or include) a perturbation with a request to predict an MOA for the perturbation. As shown in act 606 of FIG. 6, the mechanism-of-action detection system 1306 can utilize the query with a cell data repository to identify a query cell representation embedding (e.g., one or more cell representation embeddings corresponding to the perturbation associated with the query). In some cases, the mechanism-of-action detection system 1306 can generate query cell representation embedding from the query cell representation embedding in accordance with one or more implementations herein.

Furthermore, as shown in act 608 of FIG. 6, the mechanism-of-action detection system 1306 compares the query cell representation embedding with one or more mechanism of action representations (e.g., various embedding clusters for annotated cell representation embeddings associated with particular MOAs). In particular, the mechanism-of-action detection system 1306 can compare the query cell representation embedding with one or more mechanism of action representations by determining similarity measures between the embedding and the mechanism of action representations. Moreover, the mechanism-of-action detection system 1306 can utilize the similarity measures to identify one or more mechanism of action representations that are similar to the query cell representation embedding (e.g., the similarity measure satisfying a threshold similarity measure).

In some instances, the mechanism-of-action detection system 1306 can determine one or more similarity measures determined from comparisons between the query cell representation embedding with one or more mechanism of action representations. Furthermore, the mechanism-of-action detection system 1306 can compare the one or more similarity measures to a threshold similarity measure to determine if the query cell representation embedding is similar to the one or more mechanism of action representations. For example, upon a similarity measure between a query cell representation embedding and a particular mechanism of action representation satisfying a threshold similarity measure, the mechanism-of-action detection system 1306 can determine a particular MOA of the particular mechanism of action representation as a predicted MOA for the query cell representation embedding. In one or more cases, the mechanism-of-action detection system 1306 can detect multiple predicted MOAs (and confidence scores) for a query cell representation embedding in accordance with one or more implementations.

Moreover, the mechanism-of-action detection system 1306 can utilize the identified one or more mechanism of action representations that are similar to the query cell representation embedding to determine predicted mechanism of action(s) 610 for the mechanism of action query 604. In particular, the mechanism-of-action detection system 1306 can utilize MOAs associated with the one or more mechanism of action representations that are similar to the query cell representation embedding as the predicted mechanism of action(s) 610 for the mechanism of action query 604. In addition, the mechanism-of-action detection system 1306 can also generate confidence score(s) 612 for the predicted mechanism of action(s) 610 that indicate a measure of confidence that the query cell representation exhibits the predicted MOA(s) (e.g., as described in FIG. 7). In some embodiments, the mechanism-of-action detection system 1306 utilizes the similarity measurement between the query cell representation embedding and a mechanism of action representation as a prediction confidence score.

In some instances, the mechanism-of-action detection system 1306 also utilizes the MOA detection confidence score(s) 614 during prediction of MOAs. For example, the mechanism-of-action detection system 1306 can utilize the MOA detection confidence score(s) 614 to filter (e.g., remove from consideration) candidate MOAs that include an unreliable MOA representation. For instance, the mechanism-of-action detection system 1306 can compare (or check) the query cell representation embedding with MOA representations associated with MOA detection confidence scores that satisfies a threshold detection confidence score (to utilize in in the prediction of MOAs). In addition, the mechanism-of-action detection system 1306 can filter MOA representations associated with MOA detection confidence scores that do not satisfy a threshold detection confidence score. In some cases, the mechanism-of-action detection system 1306 can compare the query cell representation embedding with available MOA representations and utilize the MOA detection confidence score as part of a prediction confidence score or reliability score.

As mentioned above, the mechanism-of-action detection system 1306 can generate prediction confidence scores for predicted mechanism of actions. For instance, FIG. 7 illustrates the mechanism-of-action detection system 1306 generating a prediction confidence score for a predicted mechanism of action from an MOA representation. Indeed, FIG. 7 illustrates the mechanism-of-action detection system 1306 generating (or determining) a confidence score that indicates a measure of confidence that a query cell representation exhibits characteristics (or signals) of a predicted MOA.

For example, as shown in FIG. 7, the mechanism-of-action detection system 1306 compares a query cell representation embedding 702 to a mechanism of action representation 704 (for the predicted MOA) to generate a similarity measure 706. In particular, in some cases, the mechanism-of-action detection system 1306 compares a cluster feature of the mechanism of action representation 704 (e.g., a cluster centroid) to the query cell representation embedding 702 to generate the similarity measure 706. For instance, the similarity measure 706 can indicate a measure of the similarities (or likeness) between the mechanism of action representation 704 and the query cell representation embedding 702 within a shared feature space.

Furthermore, as shown in FIG. 7, the mechanism-of-action detection system 1306 also samples other query cell representation embeddings (e.g., sampled query cell representation embeddings 708) corresponding to a shared feature space. In some cases, the mechanism-of-action detection system 1306 randomly samples the shared feature space (or a list or repository of queries) to determine the sampled query cell representation embeddings 708. Indeed, the mechanism-of-action detection system 1306 can sample the sampled query cell representation embeddings 708 to determine whether the sampled query cell representation embeddings 708 exhibits signals (or characteristics) of the mechanism of action representation 704.

For instance, the mechanism-of-action detection system 1306 can compare the sampled query cell representation embeddings 708 to the mechanism of action 704 (e.g., a cluster centroid) to generate a plurality of similarity measures 710. Indeed, the plurality of similarity measures 710 can include a sample (or representation) of how sampled query cell representation embeddings 708 in a shared feature space relate to the mechanism of action representation 704 (e.g., to determine if the sampled query cell representation embeddings exhibit one or more meaningful signals of the mechanism of action representation 704). In some cases, the plurality of similarity measures 710 are utilized as an empirical null distribution of similarity measure scores for the particular MOA (or MOA representation). Indeed, the mechanism-of-action detection system 1306 can utilize a plurality of similarity measures 710 that includes a distribution of similarity measures between the MOA representation and sampled query cell representation embeddings to indicate a benchmark between a normal or regular similarity (e.g., no significance) versus a significant similarity that demonstrates that a particular query cell representation embedding exhibits features (or signals) of the MOA representation.

Furthermore, as shown in FIG. 7, the mechanism-of-action detection system 1306 compares the similarity measure 706 to the plurality of similarity measures 710 to generate a confidence score 712. For instance, the mechanism-of-action detection system 1306 compares the similarity measure 706 to the plurality of similarity measures 710 to determine if the query cell representation embedding 702 exhibit one or more meaningful signals of the mechanism of action representation 704 in comparison to the other sampled query cell representation embeddings 708. Indeed, the mechanism-of-action detection system 1306 can generate a comparative value as the confidence score based on the comparison between the similarity measure 706 and the plurality of similarity measures 710.

As mentioned above, the mechanism-of-action detection system 1306 can utilize a variety of similarity measures to compare query cell representation embeddings (and/or mechanism of action representations) within a shared feature space. For instance, the mechanism-of-action detection system 1306 can utilize a feature space distance measure between the embeddings as the similarity measure. As an example, the mechanism-of-action detection system 1306 can utilize cosine similarities and/or Euclidian distances between one or more query cell representation embeddings and mechanism of action representations within the shared feature space to determine a prediction confidence score.

Additionally, the mechanism-of-action detection system 1306 can generate a plurality of similarity measures (e.g., the plurality of similarity measures 710) as a distribution of similarity measures between a particular MOA representations and sampled query cell representation embeddings in the shared feature space. For example, the mechanism-of-action detection system 1306 can generate a distribution of similarity measures that represents how a sampling of query cell representation embeddings compare to a particular MOA representation. Moreover, the mechanism-of-action detection system 1306 can utilize the distribution of similarity measures for the sampled query cell representation embeddings to indicate a benchmark for identifying whether a similarity measure between a particular query cell representation embedding and the particular MOA representation represents a meaningful and/or non-meaningful indicator of similarity.

In some instances, the mechanism-of-action detection system 1306 can compare the distribution of similarity measures (e.g., the plurality of similarity measures 710) and a similarity measure (e.g., the similarity measure 706) for the particular query cell representation embedding 702 to determine a prediction confidence (e.g., as an amount of deviation from the distribution of similarity measures). For instance, the mechanism-of-action detection system 1306 can determine a deviation (or comparison) value (e.g., a standard deviation, a mean absolute deviation) between the similarity measure (between the particular query cell representation embedding and the MOA representation) to a distribution of similarity measures for the MOA representation. Moreover, the mechanism-of-action detection system 1306 can determine whether the deviation (or comparison) value (e.g., the standard deviation, mean absolute deviation) satisfies a meaningful confidence threshold (e.g., a threshold value representing a deviation value that indicates a meaningful signal). As an example, if the particular query cell representation embedding corresponds to a similarity measure that is different from a distribution of similarity measures for the MOA representation (based on satisfying a threshold deviation), the mechanism-of-action detection system 1306 can determine that the MOA representation includes data signals in the form of annotated cell representation embeddings that are meaningfully similar to the particular query cell representation embedding.

In some implementations, the mechanism-of-action detection system 1306 utilizes the deviation (or comparison) value from the particular query cell representation embedding to determine a prediction confidence score. For instance, the mechanism-of-action detection system 1306 can determine a prediction confidence score by utilizing the deviation (or comparison) value as the prediction confidence score. In some cases, the mechanism-of-action detection system 1306 can assign a prediction confidence score that increases as the deviation (or comparison) value increases (e.g., indicating a greater difference between the similarity measure of the particular query cell representation embedding to a distribution of similarity measures for the MOA representation).

Additionally, in one or more instances, the mechanism-of-action detection system 1306 can store one or more distributions of similarity measures (for comparisons with sampled query cell representation embeddings) for one or more MOA representations (generated in accordance with one or more implementations herein). Indeed, the mechanism-of-action detection system 1306 can also store one or more MOA prediction confidence scores generated for one or more MOA predictions (in response to an MOA query).

Additionally, in some embodiments, the mechanism-of-action detection system 1306 receives a list of compounds as a mechanism of action query. In response, the mechanism-of-action detection system 1306 can generate compound clusters within a shared feature space. Moreover, the mechanism-of-action detection system 1306 can predict MOAs for the compound clusters by comparing the compound clusters to MOA representations within the shared feature space (e.g., using similarity measures in accordance with one or more implementations herein). For instance, FIG. 8 illustrates the mechanism-of-action detection system 1306 generating predicted MOAs for compound clusters determined from a query list of compounds (as a mechanism of action query).

As shown in FIG. 8, the mechanism-of-action detection system 1306 receives, from a computing device 802 (e.g., a client device), a mechanism of action query 804 that includes a list of compounds. Indeed, the mechanism-of-action detection system 1306 can receive the mechanism of action query 804 as a request to generate clusters of compounds that enable MOA predictions. For instance, as shown in FIG. 8, the mechanism-of-action detection system 1306 utilizes the list of compounds (e.g., compounds 1-N) from the mechanism of action query 804 to identify cell representation embeddings that correspond to perturbations from the list of compounds (e.g., from a cell data repository).

Moreover, as shown in FIG. 8, the mechanism-of-action detection system 1306 generates compound cluster(s) 806 (within a shared feature space) utilizing the cell representation embeddings that correspond to the list of compounds. The mechanism-of-action detection system 1306 can generate the compound clusters 806 in a variety of ways. In some implementations, the mechanism-of-action detection system 1306 generates the compound clusters 806 directly from the list of compounds in a query (e.g., the query itself identifies one or more clusters or the system divides the query into clusters utilizing a clustering algorithm). In some implementations, the mechanism-of-action detection system 1306 generates the compound clusters 806 by identifying compounds similar to those identified in the mechanism of action query 804.

For example, the mechanism-of-action detection system 1306 can identify particular cell representation embeddings that correspond to particular compounds from the list of compounds. Moreover, the mechanism-of-action detection system 1306 can cluster the particular cell representation embeddings within a shared feature space utilizing a feature space analysis model (in accordance with one or more embodiments herein) based on similarity measures (e.g., cosine similarities) between the cell representation embeddings. Furthermore, upon generating the clusters of cell representation embeddings, the mechanism-of-action detection system 1306 can isolate (or identify) cell representation embedding clusters that correspond to a particular compound (based on the correspondences between the particular compounds and the particular cell representation embeddings) to generate the compound cluster(s) 806. The compound clusters can represent a grouping of compounds that have similar digital cell signals or similar perturbations within cell representations (e.g., drug molecules that cause similar perturbations).

In some cases, the mechanism-of-action detection system 1306 generates compound clusters as described in US application '707.

Additionally, as shown in FIG. 8, the mechanism-of-action detection system 1306 utilizes the compound cluster(s) 806 with the mechanism of action representations 808 to generate mechanism of action predictions for clusters within the compound clusters 810. For instance, the mechanism-of-action detection system 1306 analyzes compounds within a cluster of compounds with the MOA representations (in accordance with one or more embodiments herein) to generate MOA predictions for the compounds within the compound clusters. For instance, the mechanism-of-action detection system 1306 can compare cluster features of the compound clusters (e.g., centroids) to cluster features of MOA representations (e.g., centroids) within the shared feature space to identify similarity measures between compound clusters and the MOA representations. Then, the mechanism-of-action detection system 1306 can utilize the similarity measures between the compound clusters and the MOA representations to determine predicted MOAs for the compound clusters in accordance with one or more implementations herein.

Indeed, the mechanism-of-action detection system 1306 can generate MOA predictions for each compound in a compound cluster (in accordance with one or more embodiments herein). Indeed, as shown in FIG. 8, the mechanism-of-action detection system 1306 generate the mechanism of action predictions for compound clusters 810 to include compound clusters and associated predicted mechanism of action(s). For instance, as shown in FIG. 8, the mechanism-of-action detection system 1306 generates predicted mechanism of action(s) 812a for a compound cluster 1, predicted mechanism of action(s) 812b for a compound cluster 2, and predicted mechanism of action(s) 812n for a compound cluster N.

In addition, the mechanism-of-action detection system 1306 determines a relative frequency of the MOA associations of the compounds within a compound cluster (of the predicted MOAs from the MOA representations). For instance, the mechanism-of-action detection system 1306 can determine a number of times (e.g., a frequency) a predicted mechanism of action is associated with particular compounds (or cell representation embeddings representing compounds) within a compound cluster. Then, the mechanism-of-action detection system 1306 can utilize the determined number of times the particular mechanism of action is present for particular compounds in a compound cluster to generate a relative frequency for the particular mechanism of action prediction (in relation to the compound cluster).

Indeed, as shown in FIG. 8, the mechanism-of-action detection system 1306 determines a frequency 814a for the predicted mechanism of action(s) 812a for the compound cluster 1, determines a frequency 814b for the predicted mechanism of action(s) 812b for the compound cluster 2, and determines a frequency 814n for the predicted mechanism of action(s) 812n for the compound cluster N. As an example, if a predicted mechanism of action is predicted for three compounds in a compound cluster, the mechanism-of-action detection system 1306 can determine a relative frequency of three for the predicted mechanism of action relative to the compound cluster. In some instances, the mechanism-of-action detection system 1306 utilizes the relative frequency associated with a predicted mechanism of action as an indicator of confidence for the predicted mechanism of action for the particular compound cluster.

Furthermore, the mechanism-of-action detection system 1306 can utilize a relative frequency to determined predicted MOAs for a compound cluster. For instance, the mechanism-of-action detection system 1306 can compare a relative frequency of a predicted MOA to a threshold frequency to assign the predicted MOA a particular compound cluster. As an example, the mechanism-of-action detection system 1306 can select a predicted MOA for the particular compound cluster based on the relative frequency satisfying the threshold frequency. In some cases, the mechanism-of-action detection system 1306 can rank relative frequencies of one or more predicted MOAs to determine a selected list of predicted MOAs for a particular compound cluster (e.g., selecting the three highest relative frequencies, selecting the five highest relative frequencies, selection the highest relative frequency).

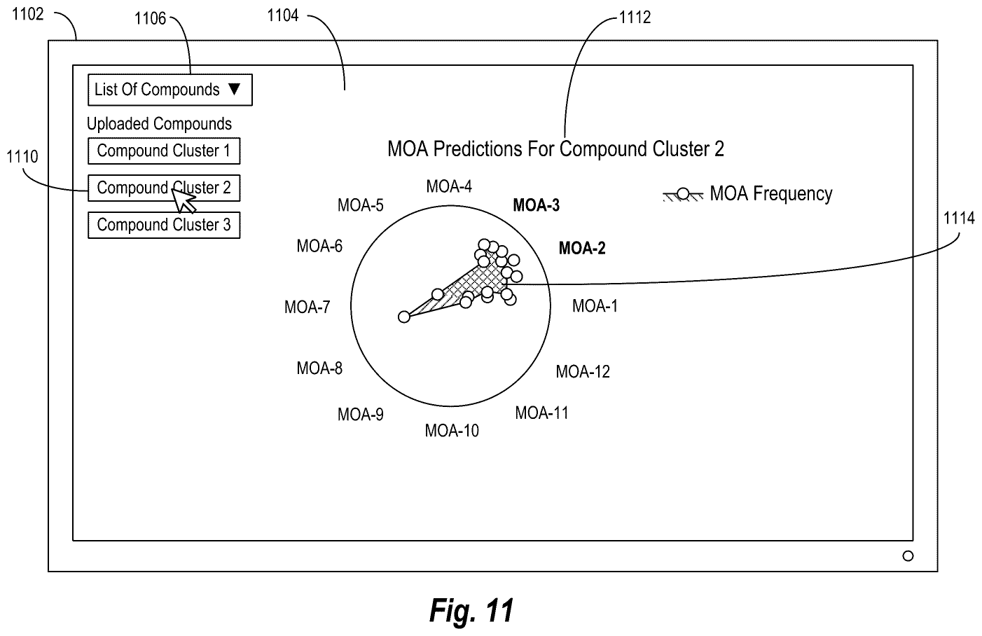

In some cases, the mechanism-of-action detection system 1306 can display one or more compound clusters in response to a mechanism of action query that includes list of compounds. For instance, the mechanism-of-action detection system 1306 can generate and list compound cluster objects or a visualization of compound clusters within a graphical user interface. In addition, upon receiving a user interaction (e.g., a user selection) of a compound cluster from the list of compound clusters (within the graphical user interface), the mechanism-of-action detection system 1306 can display predicted MOAs for the selected compound cluster (e.g., predicted in accordance with one or more implementations herein). Indeed, the mechanism-of-action detection system 1306 displaying a list of compound clusters and predicted MOAs for the compound clusters is described in greater detail below (e.g., in reference to FIG. 11).

Although one or more embodiments (and/or illustrations) describes the mechanism-of-action detection system 1306 utilizing a particular number of compounds, compound clusters, predicted MOAs, and/or frequencies, the mechanism-of-action detection system 1306 can generate and/or determine a variety compound clusters, a variety of compounds within a compound cluster, a variety of MOA predictions for a compound cluster, and various frequency determinations (or other confidence scores) for the predicted MOAs in relation to one or more compound clusters.

As mentioned above, the mechanism-of-action detection system 1306 can display one or more graphical user interfaces to display MOA representations, enable MOA query interactions, and/or display MOA query responses (e.g., predicted MOAs and/or confidence scores for the MOA predictions). For instance, FIGS. 9, 10, and 11 illustrate the mechanism-of-action detection system 1306 displaying various graphical user interfaces for mechanism of action representations, detection confidence scores, predicted mechanism of actions in response to mechanism of action queries, and/or confidence scores (in accordance with one or more implementations herein).

For instance, FIG. 9 illustrates an example of the mechanism-of-action detection system 1306 displaying an MOA representation (generated in accordance with one or more implementations herein). As shown in FIG. 9, the mechanism-of-action detection system 1306 provides, for display within a graphical user interface 904 of a client device 902, shared feature space representation 906 that includes multiple MOA representations generated in accordance with one or more implementations herein. For instance, as shown in FIG. 9, the mechanism-of-action detection system 1306 can display a shared feature space representation (e.g., the shared feature space representation) to display a representation of cell representation embeddings embedded within a shared feature space. Indeed, as illustrated in FIG. 9, the mechanism-of-action detection system 1306 can display a t-distributed Stochastic Neighbor Embedding (t-SNE) visualization as the shared feature space representation 906. Although a t-SNE visualization is demonstrated in FIG. 9, the mechanism-of-action detection system 1306 can utilize various types of visualizations to represent the MOA representations, such as, but not limited to, a uniform manifold approximation and projection (UMAP) visualization.

In addition, the mechanism-of-action detection system 1306 can also include, as part of the shared feature space representation, annotated cell representation embeddings. Indeed, as shown in FIG. 9, the mechanism-of-action detection system 1306 displays clustered embeddings that indicate MOA representations (e.g., cell representation embeddings annotated with MOAs) within the shared feature space representation 906. As an example, in reference to FIG. 9, the mechanism-of-action detection system 1306 displays an MOA representation for an MOA-1 as a cluster within the shared feature space representation 906 and various other MOA representations (e.g., MOA-2 through MOA-N) for additional MOAs as clusters within the shared feature space representation 906.

Furthermore, the mechanism-of-action detection system 1306 also displays a label (or key) section 910 within the graphical user interface 904. In particular, as shown in FIG. 9, the mechanism-of-action detection system 1306 can display a label (or key) section 910 to indicate MOA representations present within the shared feature space representation 906. For instance, in some embodiments, the mechanism-of-action detection system 1306 can display the label (or key) section 910 with mapping of colors, shapes, or other visual elements to specify specific MOA representations within the shared feature space representation 906.

In some cases, as shown in FIG. 9, the mechanism-of-action detection system 1306 can also provide, for display within the graphical user interface 904, MOA detection confidence score(s) (generated in accordance with one or more implementations herein) for the one or more MOA representations displayed within the shared feature space representation 906. For instance, as shown in FIG. 9, the mechanism-of-action detection system 1306 can display MOA detection confidence scores (e.g., detection confidence scores 1-N) for the MOA representations for MOA-1 through MOA-N.

In addition, as shown in FIG. 9, the mechanism-of-action detection system 1306 can also display, in a graphical user interface element of the graphical user interface 904, a listing 912 of one or more MOAs that are not represented as MOA representations. In some cases, the mechanism-of-action detection system 1306 can determine a list of MOAs that are not tested or not utilized by the mechanism-of-action detection system 1306 to generate an MOA representation. In some instances, the mechanism-of-action detection system 1306 can determine a list of MOAs for which a MOA representation resulted in a MOA detection confidence score that indicated that the MOA representation was unable to provide a meaningful signal for the MOA (e.g., an MOA detection confidence score that does not meet a threshold MOA detection confidence score).

Additionally, as shown in FIG. 9, the mechanism-of-action detection system 1306 can display a label (or key) section 908 to display an indication of sizes corresponding to the cell representation embeddings within the shared feature space representation 906. In particular, the mechanism-of-action detection system 1306 can display size indicators to indicate a size of cells (or other cell data) associated with the cell representation embeddings within the shared feature space representation 906.

In some cases, the mechanism-of-action detection system 1306 can display an individual MOA representation within a graphical user interface (e.g., via a shared feature space representation). Moreover, although FIG. 9 illustrates a particular number of MOA representations, the mechanism-of-action detection system 1306 can display a variety of MOA representations and/or MOA detection confidence scores in accordance with one or more implementations herein. In addition, the mechanism-of-action detection system 1306 can display one or more MOA representations separately (e.g., in different graphical user interfaces) from non-tested MOAs and/or size data.

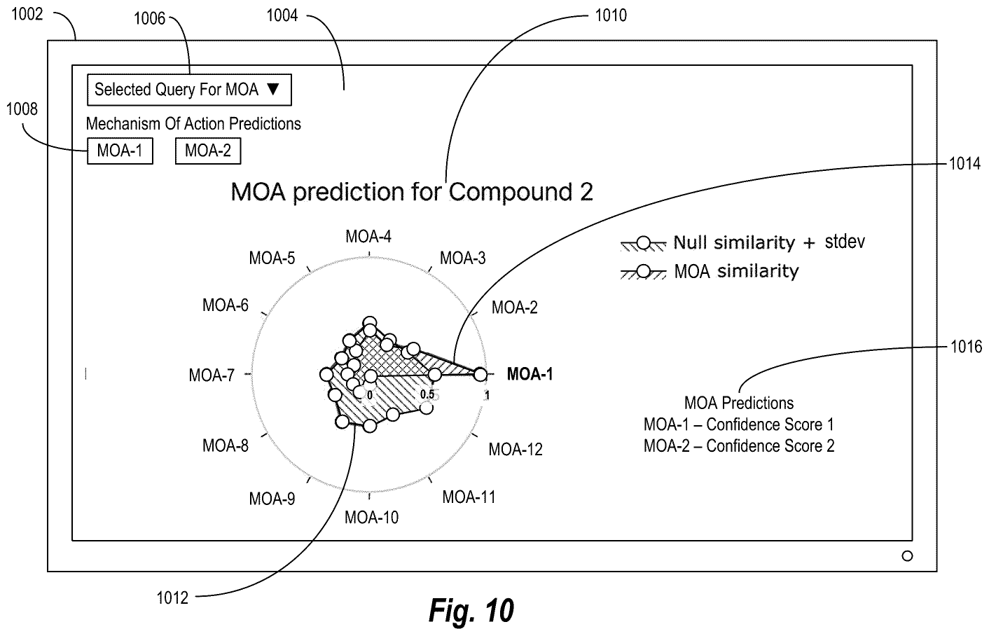

As mentioned above, the mechanism-of-action detection system 1306 can display predicted MOAs for a MOA query (in accordance with one or more implementations herein). For instance, FIG. 10 illustrates the mechanism-of-action detection system 1306 displaying an MOA query response (e.g., predicted MOAs and/or confidence scores for the MOA predictions). As shown in FIG. 10, the mechanism-of-action detection system 1306 provides, for display within a graphical user interface 1004 of a client device 1002, a selectable element 1006 to select (or provide) an MOA query. Indeed, in one or more instances, the mechanism-of-action detection system 1306 can display the selectable element 1006 utilizing various query input elements, such as, but not limited to, a dropdown list of MOA query options, a graphical user interface with selectable MOA query options, a text input box that searches for (or creates) an MOA query, and/or a button that enables voice commands to receive an MOA query. For instance, the mechanism-of-action detection system 1306 can, via user interactions with the selectable element 1006, receive an MOA query (as described above) as a request to predict MOAs for the MOA query. In some cases, the mechanism-of-action detection system 1306 can receive an upload of a cell representation (e.g., a phenomic image representing a perturbation) as an MOA query within the selectable element 1006.

In response to an MOA query via a user interaction with the selectable element 1006, the mechanism-of-action detection system 1306 can display a predicted MOA (in accordance with one or more implementations herein). For instance, as shown in FIG. 10, the mechanism-of-action detection system 1306 displays a predicted MOA listing 1008 of MOAs predicted for a particular MOA query (e.g., a query requesting MOA predictions for a compound 2). Indeed, the predicted MOA listing 1008 can include one or more MOAs predicted (e.g., MOA-1 and MOA-2) for the MOA query (in accordance with one or more implementations herein).

In addition, as shown in FIG. 10, the mechanism-of-action detection system 1306 also provides, for display within the graphical user interface 1004, a polar plot graph 1010 to display predicted MOAs for a selected query (e.g., a query requesting MOA predictions for a compound 2). As shown in FIG. 10, the mechanism-of-action detection system 1306 displays the polar plot graph 1010 to include a null distribution similarity 1012 for MOAs to indicate the plurality of similarity measures for sampled MOA queries against one or more MOA representations (e.g., as described in reference to FIG. 7). In particular, the mechanism-of-action detection system 1306 can display the null distribution similarity 1012 to represent one or more standard deviations from a null distribution (or plurality of) similarity measures between sampled MOA queries and one or more MOA representations (for the displayed MOAs).

In addition, the mechanism-of-action detection system 1306 also displays the polar plot graph 1010 to include an MOA similarity 1014 for the MOAs to indicate a similarity measure for the received MOA query against one or more MOA representations (e.g., as described in reference to FIG. 7). In particular, the mechanism-of-action detection system 1306 can display the MOA similarity 1014 to represent a similarity measure between the MOA query selected through the selectable element 1006 and the one or more MOA representations (for the displayed MOAs) in accordance with one or more implementations herein.

As shown in FIG. 10, the mechanism-of-action detection system 1306 can display the null distribution similarity 1012 and the MOA similarity 1014 overlayed within the polar plot graph 1010 to demonstrate a confidence (or a visualization of a confidence) of the predicted MOAs for the selected MOA query versus sampled MOA queries. Indeed, as shown in FIG. 10, the mechanism-of-action detection system 1306 indicates that the MOA prediction of MOA-1 is a high likelihood (or strong) prediction for the selected MOA query based on a comparison (e.g., a deviation) between the null distribution similarity 1012 and the MOA similarity 1014 for MOA-1.

As further shown in FIG. 10, the mechanism-of-action detection system 1306 can also display a confidence score generated in accordance with one or more implementations herein. For instance, as shown in FIG. 10, the mechanism-of-action detection system 1306 displays confidence scores 1016 for MOA predictions (e.g., a confidence score 1 for an MOA-1 and a confidence score 2 for an MOA-2) to a prediction confidence determined for the particular predicted MOAs in accordance with one or more implementations herein. In particular, the mechanism-of-action detection system 1306 can display a confidence score determined from a comparison between a similarity measure for the received MOA query against one or more MOA representations and a plurality of similarity measures for sampled MOA queries against one or more MOA representations (e.g., as described above in reference to FIG. 7).

Although FIG. 10 illustrates a particular number of predicted MOAs and a particular number of confidence scores for the predicted MOAs, the mechanism-of-action detection system 1306 can generate and display various numbers of predicted MOAs and/or confidence scores for an MOA query. Furthermore, the mechanism-of-action detection system 1306 can also display predicted MOAs and/or confidence scores for a variety of selected MOA queries (in accordance with one or more implementations herein).

Furthermore, in one or more instances, the mechanism-of-action detection system 1306 provides, for display within a graphical user interface, predicted MOAs for compound clusters in response to a query list of compounds (as an MOA query). For instance, FIG. 11 illustrates the mechanism-of-action detection system 1306 displaying predicted MOAs for compound clusters. For instance, the mechanism-of-action detection system 1306 can display predicted MOAs for compound clusters for a query list of compounds in accordance with one or more implementations herein (e.g., as described in FIG. 8).

As shown in FIG. 11, the mechanism-of-action detection system 1306 provides, for display within a graphical user interface 1104 of a client device 1102, a selectable element 1106 to select (or provide) a list of compounds as an MOA query. For instance, the mechanism-of-action detection system 1306 can display the selectable element 1106 utilizing various query input elements, such as, but not limited to, a dropdown list of MOA query options to select compounds, a graphical user interface with selectable MOA query options to select the compounds, a text input box that searches for (or creates) an MOA query from an input list of compounds, and/or a button that enables voice commands to receive a listing of compounds as an MOA query. For instance, the mechanism-of-action detection system 1306 can, via user interactions with the selectable element 1106, receive an MOA query (as described above) as a request to predict MOAs for the MOA query.

Additionally, the mechanism-of-action detection system 1306 can utilize the received list of compounds as the MOA query (through the selectable element 1106) to generate compound clusters and predict MOAs for the compound clusters (as described above). Moreover, as shown in FIG. 11, the mechanism-of-action detection system 1306 provides, for display within the graphical user interface 1104, a list of generated compound clusters 1110. In particular, the mechanism-of-action detection system 1306 can display the generated compound clusters 1110 to indicate clusters generated from a provided list of compounds in an MOA query. In some cases, the mechanism-of-action detection system 1306 can display a visualization of the one or more compound clusters (e.g., via a t-SNE visualization or a UMAP visualization). For instance, in some instances, the mechanism-of-action detection system 1306 can display a visualization of a compound cluster upon receiving (or detecting) a user interaction (e.g., a mouse click or touch interaction) with a compound cluster from the generated compound clusters 1110.

Moreover, as shown in FIG. 11, the mechanism-of-action detection system 1306 can receive a user interaction with a compound cluster from the displayed generated compound clusters 1110 (e.g., compound cluster 2). Based on the user interaction with a particular compound cluster from the generated compound clusters 1110, the mechanism-of-action detection system 1306 displays the MOA predictions 1112 for the selected compound cluster (e.g., compound cluster 2). Indeed, the mechanism-of-action detection system 1306 can generate the MOA predictions 1112 for the compound cluster as describe above and display the MOA predictions 1112 within the graphical user interface 1104. As shown in FIG. 11, the mechanism-of-action detection system 1306 displays the MOA predictions 1112 within a polar plot graph 1114 that indicates MOA relative frequencies (e.g., as described in FIG. 8) for one or more MOAs (e.g., MOA-1 through MOA-12). As shown in FIG. 11, the mechanism-of-action detection system 1306 indicates a prediction of MOA-2 and MOA-3 (e.g., via displaying the MOA-2 and MOA-3 indicators in bold) based on the MOA relative frequencies as described above (e.g., in reference to FIG. 8). In some cases, the mechanism-of-action detection system 1306 can display a list of predicted MOAs (e.g., a final list of predicted MOAs) based on the MOA relative frequencies as described above (e.g., in reference to FIG. 8).

Furthermore, although FIG. 11 illustrates a particular number of predicted MOAs and a particular number of cluster compounds, the mechanism-of-action detection system 1306 can generate and display various numbers of predicted MOAs and/or cluster compounds for a compound list MOA query. Furthermore, the mechanism-of-action detection system 1306 can also display various numbers of predicted MOAs for a particular compound cluster.

Although one or more embodiments illustrate the mechanism-of-action detection system 1306 utilizing polar plot graphs, the mechanism-of-action detection system 1306 can utilize various visualizations to display MOA predictions for MOA queries and/or compound list MOA queries. For instance, the mechanism-of-action detection system 1306 can utilize visualizations, such as, but not limited to, pie graphs, bar graphs, labels, data tables, and/or cloud charts.

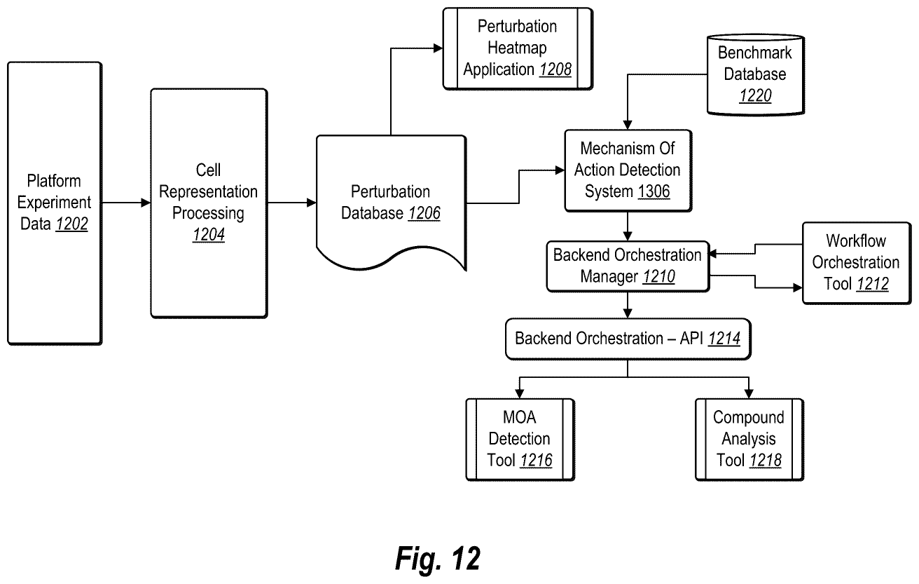

Additionally, FIG. 12 illustrates an exemplary infrastructure (or environment) in which the tech-bio exploration system 1304 and the mechanism-of-action detection system 1306 can operate. For instance, the infrastructure (or environment) illustrated in FIG. 12 includes platform experiment data 1202, cell representation processing 1204, a perturbation database 1206, and a perturbation heatmap application 1208. For instance, the tech-bio exploration system 1304 (e.g., from FIG. 13) identifies platform experiment data 1202. For example, the tech-bio exploration system 1304 can identify platform experiment data 1202 by capturing information data, such as digital images of cellular phenotypes resulting from different perturbations, determining gene sequences, and/or information in-vivo experimentation as described herein (e.g., in FIG. 13). Furthermore, as shown in FIG. 12, the tech-bio exploration system 1304 can utilize cell representation processing 1204 to generate various cell representations from the platform experiment data 1202. For instance, the mechanism-of-action detection system 1306 can generate cell representation embeddings as described in accordance with one or more implementations herein.

Furthermore, as shown in FIG. 12, the tech-bio exploration system 1304 can store cell representation embeddings and other cell data within a perturbation database 1206. In some cases, the perturbation database 1206 can include data, such as, but not limited to, cell representations, cell representation embeddings, perturbations, and/or perturbation classifications for the cell data. In addition, as shown in FIG. 12, the tech-bio exploration system 1304 can generate and store perturbation heatmaps utilizing the perturbation database 1206 for utilization in a perturbation heatmap application 1208. Indeed, the perturbation heatmap application 1208 can include perturbation heatmaps as described in US application '707.

Furthermore, the infrastructure (or environment) illustrated in FIG. 12 also includes a benchmark database 1220. In one or more instances, the benchmark database 1220 includes known biology data stored as gene-gene, gene-compound, or compound-compound relationships. In one or more instances, the benchmark database 1220 includes known biology data generated, determined, or discovered by the tech-bio exploration system 1304. Furthermore, the benchmark database 1220 can include known biology data curated from one or more external databases and/or internal user suggested (or configured) relationships.

In some instances, the mechanism-of-action detection system 1306 can also identify known MOAs from a dataset of known MOAs (as described above). For instance, the mechanism-of-action detection system 1306 can utilize a variety of datasets of known MOAs. For instance, the mechanism-of-action detection system 1306 can utilize MOA datasets and/or MOA annotation datasets as described in Cox et. al., Tales of 1,008 Small Molecules: Phenomic Profiling Through Live-Cell Imaging in a Panel of Reporter Cell Lines, Sci Rep 10, 13262 (2020), found in https://doi.org/10.1038/s41598-020-69354-8, the ChEMBL database as described in https://www.ebi.ac.uk/chembl/, and in Finan et. al., The Druggable Genome and Support for Target Identification and Validation in Drug Development, Sci Transl Med. (2017), found in https://www.science.org/doi/10.1126/scitranslmed.aag1166, each of which are incorporated by reference in their entirety herein.

In addition, as shown in the infrastructure (or environment) of FIG. 12, the mechanism-of-action detection system 1306 interacting with a backend orchestration manager 1210 to facilitate an MOA detection tool 1216 and a compound analysis tool 1218. As shown in FIG. 12, the mechanism-of-action detection system 1306 utilizes cell representation data (e.g., cell representation embeddings) and known biology data (e.g., gene-gene, gene-compound, or compound-compound relationships) to generate MOA representations, MOA detection confidence scores, MOA predictions (e.g., for MOA queries), and/or MOA prediction confidence scores in accordance with one or more implementations herein.

In addition, the mechanism-of-action detection system 1306 utilizes a backend orchestration manager 1210 with a workflow orchestration tool 1212 and a backend orchestration API 1214 to facilitate an MOA detection tool 1216 and a compound analysis tool 1218. In particular, the backend orchestration manager 1210 can schedule one or more jobs to retrieve (or identify) MOA sets, MOA representations, MOA detection confidence scores, MOA predictions, and/or MOA prediction confidence scores (generated utilizing the mechanism-of-action detection system 1306). For instance, the backend orchestration manager 1210 can enable (via the workflow orchestration tool 1212 and the backend orchestration API 1214) the MOA detection tool 1216 and/or the compound analysis tool 1218 to display one or more MOA sets, MOA representations, MOA detection confidence scores, MOA predictions, and/or MOA prediction confidence scores (generated utilizing the mechanism-of-action detection system 1306). For instance, the backend orchestration manager 1210 can receive (via the workflow orchestration tool 1212 and the backend orchestration API 1214) an MOA query and cause the mechanism-of-action detection system 1306 to determine (or generate) MOA sets, MOA representations, MOA detection confidence scores, MOA predictions, and/or MOA prediction confidence scores for the MOA query in accordance with one or more implementations herein. In one or more instances, the backend orchestration manager 1210 can utilize various frameworks and/or architectures, such as, but not limited to, Python, JavaScript, SQL, REST API, and/or Apache Kafka.

In addition, as shown in FIG. 12, the mechanism-of-action detection system 1306 facilitates an MOA detection tool 1216. For instance, the MOA detection tool 1216 can, via the mechanism-of-action detection system 1306, display data for MOA detections and/or scoring perturbations against MOAs with cell data of the tech-bio exploration system 1304 (in accordance with one or more implementations herein). In one or more instances, the MOA detection tool 1216 enables a user selection of a MOA query (e.g., selection of a perturbation, phenomap, compound) to analyze (e.g., for an MOA set). In responds to a user selection within the MOA detection tool 1216, the MOA detection tool 1216 can cause the mechanism-of-action detection system 1306 to determine (or identify) and display a predicted MOA and/or MOA prediction confidence score for the MOA query in accordance with one or more implementations. In some cases, the MOA detection tool 1216 also facilitates the display of one or more MOA representations to indicate detectable MOAs (e.g., via MOA detection confidence scores). Furthermore, the MOA detection tool 1216 facilitates one or more tools to display (or provide) interactive data between MOA queries, MOA predictions, and/or MOA representations as described above.

Furthermore, as shown in FIG. 12, the mechanism-of-action detection system 1306 facilitates a compound analysis tool 1218. For instance, the compound analysis tool 1218 can, via the mechanism-of-action detection system 1306, determine and/or display compound clusters for a list of compounds (provided as an MOA query). Furthermore, the compound analysis tool 1218 can display data for MOA detections for the compound clusters in accordance with one or more implementations herein. In some cases, the compound analysis tool 1218 can receive, from a client device, a list of compounds via a dropdown selection list and/or a data selection, such as, but not limited to, a selection of a CSV file with a listing of compounds and/or another spreadsheet file with a listing of compounds.

Additionally, the mechanism-of-action detection system 1306 can utilize MOA representations, MOA detection confidence scores, MOA predictions, and/or MOA prediction confidence scores (as described above) for various downstream tasks. For example, the mechanism-of-action detection system 1306 can utilize the MOA representations, MOA detection confidence scores, MOA predictions, and/or MOA prediction confidence scores as part of a digital drug discovery pipeline. To illustrate, in some cases, the mechanism-of-action detection system 1306 can utilize the MOA representations, MOA detection confidence scores, MOA predictions, and/or MOA prediction confidence scores generated for a compound (from one or more perturbations related to the compound) to identify candidate compounds (and compound doses) that are predicted to have certain MOAs (e.g., for in-vivo studies and/or synthesizing as pharmaceutical drugs).

In some cases, the mechanism-of-action detection system 1306 utilizes MOA representations, MOA detection confidence scores, MOA predictions, and/or MOA prediction confidence scores comparisons to determine new or unknown compounds to utilize within the tech-bio exploration system 1304 (in accordance with one or more implementations herein).

Indeed, the mechanism-of-action detection system 1306 can utilize MOA representations, MOA detection confidence scores, MOA predictions, and/or MOA prediction confidence scores within various tech-bio exploration tools of the tech-bio exploration system (e.g., as described in FIG. 13).

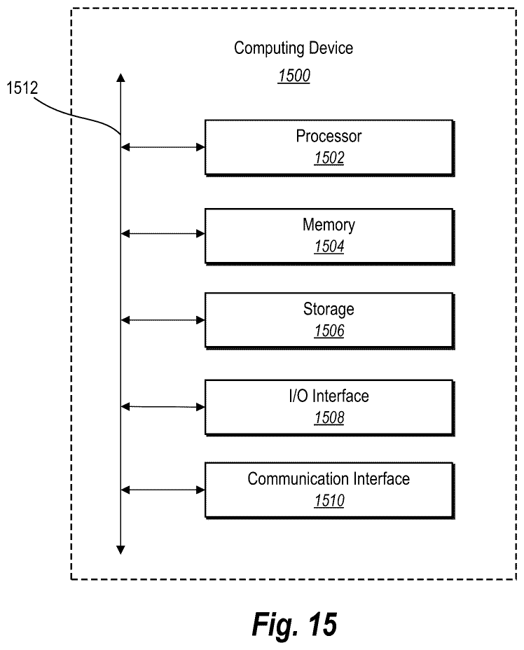

FIG. 13 illustrates a schematic diagram of a system environment in which the mechanism-of-action detection system 1306 can operate in accordance with one or more embodiments. As shown in FIG. 13, the environment includes server(s) 1302 (which includes a tech-bio exploration system 1304 and the mechanism-of-action detection system 1306), a network 1308, client device(s) 1310, and testing device(s) 1312. As further illustrated in FIG. 13, the various computing devices within the environment can communicate via the network 1308. Although FIG. 13 illustrates the mechanism-of-action detection system 1306 being implemented by a particular component and/or device within the environment, the mechanism-of-action detection system 1306 can be implemented, in whole or in part, by other computing devices and/or components in the environment (e.g., the client device(s) 1310). Additional description regarding the illustrated computing devices is provided with respect to FIG. 15 below.

As shown in FIG. 13, the server(s) 1302 can include the tech-bio exploration system 1304. In some embodiments, the tech-bio exploration system 1304 can determine, store, generate, and/or display tech-bio information including maps of biology, biology experiments from various sources, and/or machine learning tech-bio predictions. For instance, the tech-bio exploration system 1304 can analyze data signals corresponding to various treatments or interventions (e.g., compounds or biologics) and the corresponding relationships in genetics, proteomics, phenomics (i.e., cellular phenotypes), and invivomics (e.g., expressions or results within a living animal of in-vivo experiments involving chemical compounds). In one or more embodiments, the server(s) 1302 comprises a data server. In some implementations, the server(s) 1302 comprises a communication server or a web-hosting server.

For instance, the tech-bio exploration system 1304 can generate and access experimental results corresponding to gene sequences, protein shapes/folding, protein/compound interactions, phenotypes resulting from various interventions or perturbations (e.g., gene knockout sequences or compound treatments), and/or in-vivo experimentation on various treatments in living animals. By analyzing these signals (e.g., utilizing various machine learning models), the tech-bio exploration system 1304 can generate or determine a variety of predictions and inter-relationships for improving treatments/interventions.

To illustrate, the tech-bio exploration system 1304 can generate maps of biology indicating biological inter-relationships or similarities between these various input signals to discover potential new treatments. For example, the tech-bio exploration system 1304 can utilize machine learning and/or maps of biology to identify a similarity between a first gene associated with disease treatment and a second gene previously unassociated with the disease based on a similarity in resulting phenotypes from gene knockout experiments. The tech-bio exploration system 1304 can then identify new treatments based on the gene similarity (e.g., by targeting compounds the impact the second gene). Similarly, the tech-bio exploration system 1304 can analyze signals from a variety of sources (e.g., protein interactions, or in-vivo experiments) to predict efficacious treatments based on various levels of biological data.

The tech-bio exploration system 1304 can generate GUIs comprising dynamic user interface elements to convey tech-bio information and receive user input for intelligently exploring tech-bio information. Indeed, as mentioned above, the tech-bio exploration system 1304 can generate GUIs displaying different maps of biology that intuitively and efficiently express complex interactions between different biological systems for identifying improved treatment solutions. Furthermore, the tech-bio exploration system 1304 can also electronically communicate tech-bio information between various computing devices.

As shown in FIG. 13, the tech-bio exploration system 1304 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 1304 collects, manages, and transmits data across a variety of different entities, accounts, and devices. In some cases, the tech-bio exploration system 1304 is a network system that facilitates access to (and analysis of) tech-bio information within a centralized operating system. Indeed, the tech-bio exploration system 1304 can link data from different network-based research institutions to generate and analyze maps of biology.

As shown in FIG. 13, the tech-bio exploration system 1304 can include a system that comprises the mechanism-of-action detection system 1306 that can generate one or more MOA representations, MOA detection confidence scores, MOA predictions, and/or MOA prediction confidence scores in accordance with one or more implementations herein. For example, mechanism-of-action detection system 1306 can utilize cell data (e.g., maps of biology) stored, created, and/or identified by the tech-bio exploration system 1304 to generate one or more MOA representations, MOA detection confidence scores, MOA predictions, and/or MOA prediction confidence scores as described above. In addition, the mechanism-of-action detection system 1306 can utilize generated MOA representations, MOA detection confidence scores, MOA predictions, and/or MOA prediction confidence scores with one or more tech-bio exploration tools of the tech-bio exploration system.

As an example, tech-bio exploration tools can include, but are not limited to, bioactivity heatmap models as described in US application '707, ADMET prediction models and/or drug-likeness matching tools as described in US application '728, compound exploration program models as described in US application '910, digital maps of biology models as described in US application '989, and/or cell representation autoencoder models as described in US application '399.

As also illustrated in FIG. 13, the environment includes the client device(s) 1310. For example, the client device(s) 1310 may include, but is not limited to, a mobile device (e.g., smartphone, tablet) or other type of computing device, including those explained below with reference to FIG. 15. Additionally, the client device(s) 1310 can include a computing device associated with (and/or operated by) user accounts for the tech-bio exploration system 1304. Moreover, the environment can include various numbers of client devices that communicate and/or interact with the tech-bio exploration system 1304 and/or the mechanism-of-action detection system 1306.

Furthermore, in one or more implementations, the client device(s) 1310 includes a client application. The client application can include instructions that (upon execution) cause the client device(s) 1310 to perform various actions. For example, a user of a user account can interact with the client application on the client device(s) 1310 to initiate, generate, or access one or more MOA representations, MOA detection confidence scores, MOA predictions, and/or MOA prediction confidence scores in accordance with one or more implementations herein.

As further shown in FIG. 13, the environment includes the network 1308. As mentioned above, the network 1308 can enable communication between components of the environment. In one or more embodiments, the network 1308 may include a suitable network and may communicate using a various number of communication platforms and technologies suitable for transmitting data and/or communication signals, examples of which are described with reference to FIG. 15. Furthermore, although FIG. 13 illustrates computing devices communicating via the network 1308, the various components of the environment can communicate and/or interact via other methods (e.g., communicate directly).

In one or more implementations, the mechanism-of-action detection system 1306 generates and accesses one or more MOA representations, MOA detection confidence scores, MOA predictions, and/or MOA prediction confidence scores. As shown, in FIG. 13, the mechanism-of-action detection system 1306 can communicate with testing device(s) 1312 to obtain, analyze, generate, and/or store this information. For example, the tech-bio exploration system 1304 can interact with the testing device(s) 1312 that include intelligent robotic devices and camera devices for generating and capturing digital images of cellular phenotypes resulting from different perturbations (e.g., genetic knockouts or compound treatments of stem cells). Similarly, the testing device(s) 1312 can include camera devices and/or other sensors (e.g., heat or motion sensors) capturing real-time information from animals as part of in-vivo experimentation (e.g., biomarker data). The tech-bio exploration system 1304 can also interact with a variety of other testing device(s) such as devices for determining, generating, or extracting gene sequences or protein information.

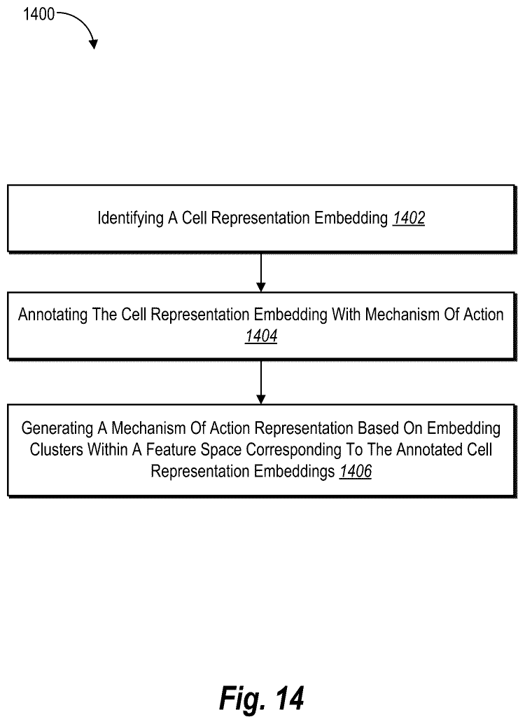

FIGS. 1-13, the corresponding text, and the examples provide a number of different systems, computer-implemented methods, and non-transitory computer readable media for deducing information for mechanism of actions (MOAs) utilizing digital signals from cell representations within a shared feature space in accordance with one or more implementations herein. In addition to the foregoing, embodiments can also be described in terms of flowcharts comprising acts for accomplishing a particular result. For example, FIG. 14 illustrate flowcharts of example sequences of acts in accordance with one or more embodiments.

While FIG. 14 illustrates acts according to some embodiments, alternative embodiments may omit, add to, reorder, and/or modify any of the acts shown in FIG. 14. The acts of FIG. 14 can be performed as part of a (computer-implemented) method. Alternatively, a non-transitory computer readable medium can comprise instructions, that when executed by one or more processors, cause a computing device to perform the acts of FIG. 14. In still further embodiments, a system can perform the acts of FIG. 14. Additionally, the acts described herein may be repeated or performed in parallel with one another or in parallel with different instances of the same or other similar acts.

For example, FIG. 14 illustrates an example series of acts for generating cell representations within a shared feature space in accordance with one or more embodiments. For example, as shown in FIG. 14, the series of acts 1400 can include an act 1402 of identifying a cell representation embedding, an act 1404 of annotating the cell representation embedding with a mechanism of action, and an act 1406 of generating a mechanism of action representation based on embedding clusters within a feature space corresponding to the annotated cell representation embeddings.

In one or more instances, the series of acts 1400 can include identifying a set of cell representation embeddings corresponding to a shared feature space and generated utilizing a machine learning model, annotating a subset of cell representation embeddings from the set of cell representation embeddings with a mechanism of action label corresponding to a mechanism of action, and generating a mechanism of action representation indicating a relationship between cell representation signals and the mechanism of action by generating an embedding cluster within the shared feature space based on the subset of cell representation embeddings with the mechanism of action label.

Furthermore, the series of acts 1400 can include identifying the set of cell representation embeddings by utilizing the machine learning model with a set of cell representations to generate the set of cell representation embeddings. For example, the series of acts 1400 can utilize a machine learning model trained to predict perturbations from cell representations or generate predicted cell representations from masked cell representations.

In addition, the series of acts 1400 can include generating the embedding cluster within the shared feature space by clustering the subset of cell representation embeddings utilizing cosine similarities. In addition, the series of acts 1400 can include generating the mechanism of action representation by determining a cluster feature from the embedding cluster that corresponds to the subset of cell representation embeddings with the mechanism of action label.

Moreover, the series of acts 1400 can include determining a mechanism of action detection confidence score for the machine learning model and the mechanism of action representation. For instance, the series of acts 1400 can include determining a similarity measure between the mechanism of action representation and a cell representation embedding within the embedding cluster. In addition, the series of acts 1400 can include identifying a plurality of similarity measures of sampled cell representation embeddings outside the embedding cluster. Moreover, the series of acts 1400 can include determining the mechanism of action detection confidence score based on the similarity measure and the plurality of similarity measures.

Additionally, the series of acts 1400 can include identifying a known perturbation corresponding to the mechanism of action. Furthermore, the series of acts 1400 can include selecting the subset of cell representation embeddings from the set of cell representation embeddings based on cell representations that correspond to the known perturbation.

In addition, the series of acts 1400 can include receiving a mechanism of action query for a perturbation. Moreover, the series of acts 1400 can include identifying, for the perturbation, a query cell representation embedding corresponding to the shared feature space and generated utilizing the machine learning model. Additionally, the series of acts 1400 can include generating a predicted mechanism of action for perturbation by comparing the query cell representation embedding with the mechanism of action representation.

Furthermore, the series of acts 1400 can include generating a confidence score for the predicted mechanism of action. Indeed, the series of acts 1400 can include determining a similarity measure between the query cell representation embedding and the mechanism of action representation of the predicted mechanism of action. In addition, the series of acts 1400 can include identifying a plurality of similarity measures between the mechanism of action representation of the predicted mechanism of action and sampled query cell representations. Moreover, the series of acts 1400 can include comparing the similarity measure to the plurality of similarity measures to determine the confidence score for the predicted mechanism of action.

Additionally, the series of acts 1400 can include providing, for display, within a graphical user interface, the predicted mechanism of action for the perturbation. Furthermore, the series of acts 1400 can include providing, for display, within the graphical user interface, the predicted mechanism of action for the perturbation, the confidence score for the predicted mechanism of action, and a visualization of the comparison between the similarity measure and the distribution of similarity measures.

Embodiments of the present disclosure may comprise or utilize a special purpose or general-purpose computer including computer hardware, such as, for example, one or more processors and system memory, as discussed in greater detail below. Implementations within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, implementations of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some implementations, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multiprocessor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Implementations of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 15 illustrates a block diagram of exemplary computing device 1500 (e.g., the server(s) 1302 and/or the client device(s) 1310) that may be configured to perform one or more of the processes described above. One will appreciate that server(s) 1302 and/or the client device(s) 1310 may comprise one or more computing devices such as computing device 1500. As shown by FIG. 15, computing device 1500 can comprise processor 1502, memory 1504, storage device 1506, I/O interface 1508, and communication interface 1510, which may be communicatively coupled by way of communication infrastructure 1512. While an exemplary computing device 1500 is shown in FIG. 15, the components illustrated in FIG. 15 are not intended to be limiting. Additional or alternative components may be used in other implementations. Furthermore, in certain implementations, computing device 1500 can include fewer components than those shown in FIG. 15. Components of computing device 1500 shown in FIG. 15 will now be described in additional detail.

In particular implementations, processor 1502 includes hardware for executing instructions, such as those making up a computer program. As an example and not by way of limitation, to execute instructions, processor 1502 may retrieve (or fetch) the instructions from an internal register, an internal cache, memory 1504, or storage device 1506 and decode and execute them. In particular implementations, processor 1502 may include one or more internal caches for data, instructions, or addresses. As an example and not by way of limitation, processor 1502 may include one or more instruction caches, one or more data caches, and one or more translation lookaside buffers (TLBs). Instructions in the instruction caches may be copies of instructions in memory 1504 or storage device 1506.

Memory 1504 may be used for storing data, metadata, and programs for execution by the processor(s). Memory 1504 may include one or more of volatile and non-volatile memories, such as Random Access Memory (“RAM”), Read Only Memory (“ROM”), a solid state disk (“SSD”), Flash, Phase Change Memory (“PCM”), or other types of data storage. Memory 1504 may be internal or distributed memory.

Storage device 1506 includes storage for storing data or instructions. As an example and not by way of limitation, storage device 1506 can comprise a non-transitory storage medium described above. Storage device 1506 may include a hard disk drive (HDD), a floppy disk drive, flash memory, an optical disc, a magneto-optical disc, magnetic tape, or a Universal Serial Bus (USB) drive or a combination of two or more of these. Storage device 1506 may include removable or non-removable (or fixed) media, where appropriate. Storage device 1506 may be internal or external to computing device 1500. In particular implementations, storage device 1506 is non-volatile, solid-state memory. In other implementations, Storage device 1506 includes read-only memory (ROM). Where appropriate, this ROM may be a mask programmed ROM, programmable ROM (PROM), erasable PROM (EPROM), electrically erasable PROM (EEPROM), electrically alterable ROM (EAROM), or flash memory or a combination of two or more of these.

I/O interface 1508 allows a user to provide input to, receive output from, and otherwise transfer data to and receive data from computing device 1500. I/O interface 1508 may include a mouse, a keypad or a keyboard, a touch screen, a camera, an optical scanner, network interface, modem, other known I/O devices or a combination of such I/O interfaces. I/O interface 1508 may include one or more devices for presenting output to a user, including, but not limited to, a graphics engine, a display (e.g., a display screen), one or more output drivers (e.g., display drivers), one or more audio speakers, and one or more audio drivers. In certain implementations, I/O interface 1508 is configured to provide graphical data to a display for presentation to a user. The graphical data may be representative of one or more graphical user interfaces and/or any other graphical content as may serve a particular implementation.

Communication interface 1510 can include hardware, software, or both. In any event, communication interface 1510 can provide one or more interfaces for communication (such as, for example, packet-based communication) between computing device 1500 and one or more other computing devices or networks. As an example and not by way of limitation, communication interface 1510 may include a network interface controller (NIC) or network adapter for communicating with an Ethernet or other wire-based network or a wireless NIC (WNIC) or wireless adapter for communicating with a wireless network, such as a WI-FI.

Additionally or alternatively, communication interface 1510 may facilitate communications with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, communication interface 1510 may facilitate communications with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination thereof.

Additionally, communication interface 1510 may facilitate communications various communication protocols. Examples of communication protocols that may be used include, but are not limited to, data transmission media, communications devices, Transmission Control Protocol (“TCP”), Internet Protocol (“IP”), File Transfer Protocol (“FTP”), Telnet, Hypertext Transfer Protocol (“HTTP”), Hypertext Transfer Protocol Secure (“HTTPS”), Session Initiation Protocol (“SIP”), Simple Object Access Protocol (“SOAP”), Extensible Mark-up Language (“XML”) and variations thereof, Simple Mail Transfer Protocol (“SMTP”), Real-Time Transport Protocol (“RTP”), User Datagram Protocol (“UDP”), Global System for Mobile Communications (“GSM”) technologies, Code Division Multiple Access (“CDMA”) technologies, Time Division Multiple Access (“TDMA”) technologies, Short Message Service (“SMS”), Multimedia Message Service (“MMS”), radio frequency (“RF”) signaling technologies, Long Term Evolution (“LTE”) technologies, wireless communication technologies, in-band and out-of-band signaling technologies, and other suitable communications networks and technologies.

Communication infrastructure 1512 may include hardware, software, or both that couples components of computing device 1500 to each other. As an example and not by way of limitation, communication infrastructure 1512 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
identifying a set of cell representation embeddings corresponding to a shared feature space;
annotating a subset of cell representation embeddings from the set of cell representation embeddings with a mechanism of action label corresponding to a mechanism of action; and
generating a mechanism of action representation for the mechanism of action by generating an embedding cluster within the shared feature space based on the subset of cell representation embeddings with the mechanism of action label.

2. The computer-implemented method of claim 1, further comprising generating the set of cell representation embeddings utilizing a machine learning model trained to predict perturbations from cell representations or generate predicted cell representations from masked cell representations.

3. The computer-implemented method of claim 1, further comprising generating the embedding cluster within the shared feature space by clustering the subset of cell representation embeddings utilizing cosine similarities.

4. The computer-implemented method of claim 1, further comprising generating the mechanism of action representation by determining a cluster feature from the embedding cluster that corresponds to the subset of cell representation embeddings with the mechanism of action label.

5. The computer-implemented method of claim 1, further comprising determining a mechanism of action detection confidence score for the mechanism of action representation by:
determining a similarity measure between the mechanism of action representation and a cell representation embedding within the embedding cluster;
identifying a plurality of similarity measures of sampled cell representation embeddings outside the embedding cluster; and
determining the mechanism of action detection confidence score based on the similarity measure and the plurality of similarity measures.

6. The computer-implemented method of claim 1, further comprising:
identifying a known perturbation corresponding to the mechanism of action; and
selecting the subset of cell representation embeddings from the set of cell representation embeddings based on cell representations that correspond to the known perturbation.

7. The computer-implemented method of claim 1, further comprising:
receiving a mechanism of action query for a perturbation;
identifying, for the perturbation, a query cell representation embedding corresponding to the shared feature space; and
generating a predicted mechanism of action for perturbation by comparing the query cell representation embedding with the mechanism of action representation.

8. The computer-implemented method of claim 7, further comprising generating a confidence score for the predicted mechanism of action by:
determining a similarity measure between the query cell representation embedding and the mechanism of action representation of the predicted mechanism of action;
identifying a plurality of similarity measures between the mechanism of action representation of the predicted mechanism of action and sampled query cell representations; and
comparing the similarity measure to the plurality of similarity measures to determine the confidence score for the predicted mechanism of action.

9. The computer-implemented method of claim 7, further comprising providing, for display, within a graphical user interface, the predicted mechanism of action for the perturbation.

10. A system comprising:
at least one processor; and
at least one non-transitory computer-readable storage medium storing instructions that, when executed by the at least one processor, cause the system to:
identify a set of cell representation embeddings corresponding to a shared feature space;
annotate a subset of cell representation embeddings from the set of cell representation embeddings with a mechanism of action label corresponding to a mechanism of action; and
generate a mechanism of action representation for the mechanism of action by generating an embedding cluster within the shared feature space based on the subset of cell representation embeddings with the mechanism of action label.

11. The system of claim 10, wherein the instructions cause the system to generate the set of cell representation embeddings utilizing a machine learning model trained to predict perturbations from cell representations or generate predicted cell representations from masked cell representations.

12. The system of claim 10, wherein the instructions cause the system to determine a mechanism of action detection confidence score for the mechanism of action representation by:
determining a similarity measure between the mechanism of action representation and a cell representation embedding within the embedding cluster;
identifying a plurality of similarity measures of sampled cell representation embeddings outside the embedding cluster; and
determine the mechanism of action detection confidence score based on the similarity measure and the plurality of similarity measures.

13. The system of claim 10, wherein the instructions cause the system to:
receive a mechanism of action query for a perturbation;
identify, for the perturbation, a query cell representation embedding corresponding to the shared feature space; and
generate a predicted mechanism of action for perturbation by comparing the query cell representation embedding with the mechanism of action representation.

14. The system of claim 13, wherein the instructions cause the system to generate a confidence score for the predicted mechanism of action by:
determining a similarity measure between the query cell representation embedding and the mechanism of action representation of the predicted mechanism of action;
identifying a plurality of similarity measures between the mechanism of action representation of the predicted mechanism of action and sampled query cell representations; and
comparing the similarity measure to the plurality of similarity measures to determine the confidence score for the predicted mechanism of action.

15. The system of claim 14, wherein the instructions cause the system to provide, for display, within a graphical user interface, the predicted mechanism of action for the perturbation, the confidence score for the predicted mechanism of action, and a visualization of a comparison between the similarity measure and the plurality of similarity measures.

16. A non-transitory computer-readable medium storing instructions that, when executed by at least one processor, cause a computing device to:
identify a set of cell representation embeddings corresponding to a shared feature space;
annotate a subset of cell representation embeddings from the set of cell representation embeddings with a mechanism of action label corresponding to a mechanism of action; and
generate a mechanism of action representation for the mechanism of action by generating an embedding cluster within the shared feature space based on the subset of cell representation embeddings with the mechanism of action label.

17. The non-transitory computer-readable medium of claim 16, wherein the instructions cause the computing device to generate the set of cell representation embeddings utilizing a machine learning model trained to predict perturbations from cell representations or generate predicted cell representations from masked cell representations.

18. The non-transitory computer-readable medium of claim 16, wherein the instructions cause the computing device to determine a mechanism of action detection confidence score for the mechanism of action representation by:
determining a similarity measure between the mechanism of action representation and a cell representation embedding within the embedding cluster;
identifying a plurality of similarity measures of sampled cell representation embeddings outside the embedding cluster; and
determine the mechanism of action detection confidence score based on the similarity measure and the plurality of similarity measures.

19. The non-transitory computer-readable medium of claim 16, wherein the instructions cause the computing device to:
receive a mechanism of action query for a perturbation;
identify, for the perturbation, a query cell representation embedding corresponding to the shared feature space; and
generate a predicted mechanism of action for perturbation by comparing the query cell representation embedding with the mechanism of action representation.

20. The non-transitory computer-readable medium of claim 19, wherein the instructions cause the computing device to generate a confidence score for the predicted mechanism of action by:
determining a similarity measure between the query cell representation embedding and the mechanism of action representation of the predicted mechanism of action;
identifying a plurality of similarity measures between the mechanism of action representation of the predicted mechanism of action and sampled query cell representations; and
comparing the similarity measure to the plurality of similarity measures to determine the confidence score for the predicted mechanism of action.
